SEQUENCE LISTING (67 sequences)

SEQ ID NO: 1 - protein, 1368 aa, Streptococcus pyogenes [sequence omitted]

SEQ ID NO: 2 - protein, 497 aa, Syntrophomonas palmitatica
MGESVKAIKLKILDMFLDPECTKQDDNWRKDLSTMSRFCAEAGNMCLRDLYNYFSMPKEDRISSKDLYNAMYHKTKLLHPELPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 3 - protein, 497 aa, synthetic construct
  source 1..497: note = synthetic construct
MGESVKAIKLKILDMFLDPECTKQDDNWRKDLSTMSRFCGEAGNMCLRDLYNYFSMPKEDRISSKDLYNAMYHKTKLLHPGLPGKVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 4 - protein, 497 aa, synthetic construct
  source 1..497: note = synthetic construct
MGESVKAIKLKILDMFLDPECTKQDDNWRKDLSTMSRFCGEAGNSCLRDLYNYDSMPKEDRISSKDLYNAMYHKTKLLHPELPGQVKNQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYKQGQLQIKKDHQGKWYCIIPYTFPTHEHVLDPDKVMGVDLGVAKAVYWAFNSSYKRGCIDGGEIEHFRKMIRARRVSIQNQIKHSGDARKGHGRKRALKPIETLSEKESNFRDTINFRYANRIVEAAIKQGCGTIQIENLEGIADRTGSKFLKNWPYYDLQTKIVNKAKEHGITVVAINPQYTSQRCSMCGYIEKTNRSSQAVFECKQCGYGSRTNCINCRHVQVSGDVCEECGGIVKKENVNADYNAAKNISTPYIDQIIMEKCLELGIPYRSITCKECDHIQASGNTCEVCGSTNILKPKKIRKAK

SEQ ID NO: 5 - protein, 422 aa, Acidibacillus sulfuroxidans
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 6 - protein, 422 aa, synthetic construct
  source 1..422: note = synthetic construct
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDYPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLTQGKYAGGARGGHGRDKRIKPIEQLRDKIPNFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 7 - protein, 98 aa, Arabidopsis thaliana
TCAKDIQKAAAEAALAFQDETCDTTTTNHGLDMEETMVEAIYTPEQSEGAFYMDEETMFGMPTLLDNMAEGMLLPPPSVQWNHNYDGEGDGDVSLWSY

SEQ ID NO: 8 - protein, 18 aa, Agrobacterium tumefaciens
KRPRDRHDGELGGRKRAR

SEQ ID NO: 9 - DNA, 896 bp, Zea mays
  source 1..896: mol_type = genomic DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctt

SEQ ID NO: 10 - DNA, 82 bp, Zea mays
  source 1..82: mol_type = genomic DNA
tccccaacctcgtgttgttcggagcgcacacacacacaaccagatctcccccaaatccacccgtcggcacctccgcttcaag

SEQ ID NO: 11 - DNA, 1013 bp, Zea mays
  source 1..1013: mol_type = genomic DNA
gtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 12 - DNA, 21 bp, synthetic construct
  source 1..21: mol_type = other DNA; note = synthetic construct
ccgaagaagaagcgcaaggtg

SEQ ID NO: 13 - DNA, 189 bp, Solanum tuberosum
  source 1..189: mol_type = genomic DNA
gtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcag

SEQ ID NO: 14 - DNA, 1680 bp, synthetic construct
  source 1..1680: mol_type = other DNA; note = synthetic construct
atgggcgagagcgtcaaggcaataaaattaaagatcctcgacatgttcctcgaccccgagtgcaccaagcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggacgacaactggcgcaaggacctcagcaccatgagccgcttctgcggcgaggccggcaacatgtgcctcagggacctctacaactacttcagcatgcccaaggaggaccgcatcagctccaaggacttatataacgccatgtaccataaaactaagctcctccaccccggcctccccgggaaggtgaagaaccaaatcgtcaaccacgccaaggacgtctggaagcgcaacgccaagctcatctaccgcaaccaaatcagcatgcccacatataagatcaccaccgcccccatccgcctccaaaataacatctacaaattaataaaaaataagaacaaatacataatcgacgtccagctctacagcaaggagtacagcaaggacagcggcaagggcacccacaggtacttcctcgtcgccgtcagggacagcagcaccaggatgatcttcgacaggatcatgagcaaggaccacatcgacagcagcaagagctacaagcagggccagctccaaatcaagaaggaccaccagggcaagtggtactgcatcatcccctatacattccccacccacgaacacgtcctcgaccccgacaaggtcatgggcgtcgacctcggggtggctaaggccgtctactgggctttcaacagcagctataaaagaggctgcatcgacggcggcgagatcgagcacttcaggaagatgatcagggcccggcgcgtcagcatccagaatcaaatcaaacacagcggcgacgcccgcaagggccacggcaggaagagggccctcaagcccatcgaaaccctcagcgagaaggagagcaacttccgcgacacaataaacttcaggtacgccaacaggatcgtcgaggccgctatcaagcagggctgcggcaccatccagatcgagaacctcgagggcatcgctgacaggaccggcagcaagttcctcaagaactggccctactacgacctccagaccaagatcgtcaataaagccaaggagcacggcatcaccgtcgtcgcaataaacccccagtatacatcccagcgctgcagcatgtgcggctacatcgagaaaaccaacaggagcagccaggccgtgttcgagtgcaagcagtgcggctacggcagccgcaccaactgcatcaactgcaggcacgtccaagtctccggcgacgtctgcgaggagtgcggcggcatcgtcaagaaggagaacgtcaacgccgactacaacgccgccaagaacatcagcaccccctacatcgaccagataataatggagaagtgcctcgagctcggcatcccctaccgctccatcacctgcaaggagtgcggccacatccaggctagcggcaacacctgcgaggtctgcggcagcaccaacatcctcaaaccaaagaagatccgcaaggcaaaa

SEQ ID NO: 15 - DNA, 1680 bp, synthetic construct
  source 1..1680: mol_type = other DNA; note = synthetic construct
atgggcgagtccgtgaaggccatcaagctcaagatcctggacatgttcctcgacccggagtgcaccaagcaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggacgacaactggaggaaggacctctccaccatgagccgcttctgcggcgaggctggcaacagctgcctcagggacctgtacaactacgactccatgccgaaggaggaccgcatctccagcaaggacctctacaacgcgatgtaccacaagacgaagctcctgcaccccgagctgcccggccaggtgaagaaccagatcgtcaaccacgccaaggacgtgtggaagaggaacgcgaagctgatctaccgcaaccagatctccatgccgacgtacaagatcaccacggcccccatcaggctccagaacaacatctacaagctgatcaagaacaagaacaagtacatcatcgacgtccagctctacagcaaggagtactccaaggacagcggcaagggcacccaccgctacttcctggtggccgtccgcgactccagcacgaggatgatcttcgaccgcatcatgtccaaggaccacatcgactccagcaagagctacaagcagggccagctccagatcaagaaggaccaccagggcaagtggtactgcatcatcccgtacaccttcccgacccacgagcacgtgctcgacccggacaaggtcatgggcgtggacctcggcgtggctaaggccgtctactgggccttcaactccagctacaagcggggctgcatcgacggcggcgagatcgagcacttccgcaagatgatcagggcgcgcagggtctccatccagaaccagatcaagcactcgggcgacgctaggaagggccacggccgcaagagggcgctcaagcccatcgagaccctgtccgagaaggagagcaacttccgcgacacgatcaacttccgctacgccaacaggatcgtggaggcggcgatcaagcagggctgcggcaccatccagatcgagaacctggagggcatcgccgacaggacgggcagcaagttcctcaagaactggccgtactacgacctgcagaccaagatcgtcaacaaggccaaggagcacggcatcaccgtggtcgcgatcaacccccagtacacgtcccagaggtgcagcatgtgcggctacatcgagaagaccaaccgctccagccaggccgtgttcgagtgcaagcagtgcggctacggctccaggacgaactgcatcaactgccgccacgtgcaggtcagcggcgacgtctgcgaggagtgcggtggcatcgtgaagaaggagaacgtcaacgcggactacaacgccgcgaagaacatctccaccccgtacatcgaccagatcatcatggagaagtgcctggagctgggcatcccctacaggagcatcacgtgcaaggagtgcgaccacatccaggcctccggcaacacctgcgaggtgtgcggcagcacgaacatcctcaagcccaagaagatccgcaaggcgaag

SEQ ID NO: 16 - DNA, 57 bp, synthetic construct
  source 1..57: mol_type = other DNA; note = synthetic construct
aagagaccacgggaccgccacgatggcgagctgggaggccgcaagcgggcaaggtag

SEQ ID NO: 17 - DNA, 294 bp, synthetic construct
  source 1..294: mol_type = other DNA; note = synthetic construct
acgtgcgccaaggacatccaaaaagcagccgcggaggcggcactcgcctttcaggacgagacctgcgacaccaccaccaccaaccacggcctcgacatggaggagaccatggtcgaagccatctacacccccgagcagagcgagggcgccttctacatggacgaagagacaatgttcggcatgccaaccctcctcgacaacatggccgaaggcatgctgctgccgcccccgagcgtccagtggaaccacaactacgacggcgaaggcgacggggatgtgtccctgtggagctac

SEQ ID NO: 18 - DNA, 910 bp, Zea mays
  source 1..910: mol_type = genomic DNA
gtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgac

SEQ ID NO: 19 - DNA, 4293 bp, synthetic construct
  source 1..4293: mol_type = other DNA; note = synthetic construct
atggacaagaagtacagcatcggcctcgacatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgaccacatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagac

SEQ ID NO: 20 - DNA, 1001 bp, synthetic construct
  source 1..1001: mol_type = other DNA; note = synthetic construct
tgagagtacaatgatgaacctagattaatcaatgccaaagtctgaaaaatgcaccctcagtctatgatccagaaaatcaagattgcttgaggccctgttcggttgttccggattagagccccggattaattcctagccggattacttctctaatttatatagattttgatgagctggaatgaatcctggcttattccggtacaaccgaacaggccctgaaggataccagtaatcgctgagctaaattggcatgctgtcagagtgtcagtattgcagcaaggtagtgagataaccggcatcatggtgccagtttgatggcaccattagggttagagatggtggccatgggcgcatgtcctggccaactttgtatgatatatggcagggtgaataggaaagtaaaattgtattgtaaaaagggatttcttctgtttgttagcgcatgtacaaggaatgcaagttttgagcgagggggcatcaaagatctggctgtgtttccagctgtttttgttagccccatcgaatccttgacataatgatcccgcttaaataagcaacctcgcttgtatagttccttgtgctctaacacacgatgatgataagtcgtaaaatagtggtgtccaaagaatttccaggcccagttgtaaaagctaaaatgctattcgaatttctactagcagtaagtcgtgtttagaaattatttttttatataccttttttccttctatgtacagtaggacacagtgtcagcgccgcgttgacggagaatatttgcaaaaaagtaaaagagaaagtcatagcggcgtatgtgccaaaaacttcgtcacagagagggccataagaaacatggcccacggcccaatacgaagcaccgcgacgaagcccaaacagcagtccgtaggtggagcaaagcgctgggtaatacgcaaacgttttgtcccaccttgactaatcacaagagtggagcgtaccttataaaccgagccgcaagcaccgaattg

SEQ ID NO: 21 - DNA, 20 bp, Zea mays
  source 1..20: mol_type = genomic DNA
cataatgaggatcgaggatg

SEQ ID NO: 22 - DNA, 13 bp, Zea mays
  source 1..13: mol_type = genomic DNA
aagtttaggccgg

SEQ ID NO: 23 - DNA, 14 bp, Zea mays
  source 1..14: mol_type = genomic DNA
agccctgagatcgc

SEQ ID NO: 24 - DNA, 13 bp, Zea mays
  source 1..13: mol_type = genomic DNA
aatctctgatgct

SEQ ID NO: 25 - DNA, 14 bp, Zea mays
  source 1..14: mol_type = genomic DNA
ctaggtatgtacgt

SEQ ID NO: 26 - DNA, 13 bp, Zea mays
  source 1..13: mol_type = genomic DNA
acgacggagaggc

SEQ ID NO: 27 - DNA, 16 bp, Zea mays
  source 1..16: mol_type = genomic DNA
aaagctaatgtgcgtg

SEQ ID NO: 28 - DNA, 18 bp, Zea mays
  source 1..18: mol_type = genomic DNA
ggatcaatgatcggggtc

SEQ ID NO: 29 - DNA, 18 bp, Zea mays
  source 1..18: mol_type = genomic DNA
tcggggagatggggcttt

SEQ ID NO: 30 - DNA, 20 bp, Zea mays
  source 1..20: mol_type = genomic DNA
tggctagctagcgcgcgaga

SEQ ID NO: 31 - DNA, 16 bp, Zea mays
  source 1..16: mol_type = genomic DNA
catgccacgagctttc

SEQ ID NO: 32 - DNA, 77 bp, synthetic construct
  source 1..77: mol_type = other DNA; note = synthetic construct
gttttagagctagaaatagcaagttaaaataaggctagtccgttatcaacttgaaaaagtggcaccgagtcggtgct

SEQ ID NO: 33 - skipped

SEQ ID NO: 34 - DNA, 190 bp, synthetic construct
  source 1..190: mol_type = other DNA; note = synthetic construct
gatttactctgtttcgcgcgccagggcagttaggtgccctaaaagagcgaagtggccgaaaggaaaggctaacgcttctctaacgctacggcgaccttggcgaaatgccatcaataccacgcggcccgaaagggttcgcgcgaaactgagtaatgaaagtcgcatcttgcgtaagcgcgtggattgaaac

SEQ ID NO: 35 - DNA, 145 bp, synthetic construct
  source 1..145: mol_type = other DNA; note = synthetic construct
tctattcgtcggttcagcgacgataagccgagaagtgccaataaaactgttaagtggtttggtaacgctcggtaaggtagccaaaaggctgaaactccgtgcacaaagaccgcacggacgcttcacagaaatgtggagtgtgaac

SEQ ID NO: 36 - DNA, 20 bp, Zea mays
  source 1..20: mol_type = genomic DNA
cacagggagggagcaaacaa

SEQ ID NO: 37 - DNA, 12 bp, Zea mays
  source 1..12: mol_type = genomic DNA
gggcaataattc

SEQ ID NO: 38 - DNA, 12 bp, Zea mays
  source 1..12: mol_type = genomic DNA
ggaatctctctc

SEQ ID NO: 39 - DNA, 12 bp, Zea mays
  source 1..12: mol_type = genomic DNA
taacagcgcaag

SEQ ID NO: 40 - DNA, 12 bp, Zea mays
  source 1..12: mol_type = genomic DNA
aggttatgtgat

SEQ ID NO: 41 - DNA, 18 bp, Zea mays
  source 1..18: mol_type = genomic DNA
ggtcatcgcaccgcacgc

SEQ ID NO: 42 - DNA, 18 bp, Zea mays
  source 1..18: mol_type = genomic DNA
gggcaataattcaggcgc

SEQ ID NO: 43 - DNA, 18 bp, Zea mays
  source 1..18: mol_type = genomic DNA
cggaatctctctcgtctt

SEQ ID NO: 44 - DNA, 18 bp, Zea mays
  source 1..18: mol_type = genomic DNA
cgctgttagaacctggct

SEQ ID NO: 45 - DNA, 18 bp, Zea mays
  source 1..18: mol_type = genomic DNA
caggttatgtgatgatgt

SEQ ID NO: 46 - DNA, 68 bp, Hepatitis Delta Virus
  source 1..68: mol_type = genomic DNA
ggccggcatggtcccagcctcctcgctggcgccggctgggcaacatgcttcggcatggcgaatgggac

SEQ ID NO: 47 - DNA, 1455 bp, unidentified
  source 1..1455: mol_type = other DNA; note = synthetic construct
atgatcaaggtctaccgctacgagatcgtcaagcccctcgacctcgactggaaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggagttcggcaccatcctcaggcagctccagcaggaaaccaggttcgccctcaacaaggccacccagctcgcctgggagtggatgggcttcagcagcgactacaaggacaaccacggcgagtaccccaagagcaaggacatcctcggctacaccaacgtccacggctacgcctaccacaccatcaaaaccaaagcatatcgcctcaacagcggcaacctcagccagaccatcaagcgcgccaccgaccgcttcaaagcatatcagaaggagatcctcaggggcgacatgagcatccccagctacaagcgcgactaccccctcgacctcattaaagagaacatcagcgtcaaccgcatgaaccacggcgactacatcgccagcctcagcctcctgtccaaccccgccaagcaggagatgaacgtcaagcgcaagatcagcgtcatcatcatcgtcaggggcgccggcaagaccatcatggaccgcatcctcagcggcgagtaccaggtcagcgccagccaaataattcacgacgaccgcaagaacaagtggtatctcaacatcagctacgacttcgagccccagaccagggtcctcgacctcaataaaataatgggcatcgccctcggcgtcgccgtcgccgtctacatggccttccagcacacccccgccaggtacaagctcgagggcggcgagatcgagaacttcaggaggcaggtcgagagccgccgcatctccatgctcacccagggcaagtacgccggcggcgccaggggcggccacggcagggataaaagaataaaacccatcgagcagctccgcgataaaattccaaacttccgcgacaccaccaaccaccgctacagccgctacatcgtcgacatggcaattaaagagggctgcggcaccatccagatggaggacctcaccaacatccgcgacatcggcagccgcttcctccagaactggacctactacgacctccagcagaagatcatctacaaggccgaggaggccggaattaaagtcataaagatcgacccccagtacaccagccagaggtgcagcgagtgcggcaacatcgacagcggcaacaggatcggccaggccatcttcaagtgccgcgcctgcggctacgaggccaacgccgactacaacgccgcccgcaacatcgccatccccaacatcgacaagatcatcgccgagagcatcaag

SEQ ID NO: 48 - DNA, 4293 bp, synthetic construct
  source 1..4293: mol_type = other DNA; note = synthetic construct
atggacaagaagtacagcatcggcctcgccatcggcaccaactcggtgggctgggccgtcatcacggacgaatataaggtcccgtcgaagaagttcaaggtcctcggcaatacagaccgccacagcatcaagaaaaacttgatcggcgccctcctgttcgatagcggcgagaccgcggaggcgaccaggctcaagaggaccgccaggagacggtacactaggcgcaagaacaggatctgctacctgcaggagatcttcagcaacgagatggcgaaggtggacgactccttcttccaccgcctggaggaatcattcctggtggaggaggacaagaagcatgagcggcacccaatcttcggcaacatcgtcgacgaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgtgcaggtggcctaccacgagaagtacccgacaatctaccacctccggaagaaactggtggacagcacagacaaggcggacctccggctcatctaccttgccctcgcgcatatgatcaagttccgcggccacttcctcatcgagggcgacctgaacccggacaactccgacgtggacaagctgttcatccagctcgtgcagacgtacaatcaactgttcgaggagaaccccataaacgctagcggcgtggacgccaaggccatcctctcggccaggctctcgaaatcaagaaggctggagaaccttatcgcgcagttgccaggcgaaaagaagaacggcctcttcggcaaccttattgcgctcagcctcggcctgacgccgaacttcaaatcaaacttcgacctcgcggaggacgccaagctccagctctcaaaggacacctacgacgacgacctcgacaacctcctggcccagataggagaccagtacgcggacctcttcctcgccgccaagaacctctccgacgctatcctgctcagcgacatccttcgggtcaacaccgaaattaccaaggcaccgctgtccgccagcatgattaaacgctacgacgagcaccatcaggacctcacgctgctcaaggcactcgtccgccagcagctccccgagaagtacaaggagatcttcttcgaccaatcaaaaaacggctacgcgggatatatcgacggcggtgccagccaggaagagttctacaagttcatcaaaccaatcctggagaagatggacggcaccgaggagttgctggtcaagctcaacagggaggacctcctcaggaagcagaggaccttcgacaacggctccatcccgcatcagatccacctgggcgaactgcatgccatcctgcggcgccaggaggacttctacccgttcctgaaggataaccgggagaagatcgagaagatcttgacgttccgcatcccatactacgtgggcccgctggctcgcggcaactcccggttcgcctggatgacccggaagtcggaggagaccatcacaccctggaactttgaggaggtggtcgataagggcgctagcgctcagagcttcatcgagcgcatgaccaacttcgataaaaacctgcccaatgaaaaagtcctccccaagcactcgctgctctacgagtacttcaccgtgtacaacgagctcaccaaggtcaaatacgtcaccgagggcatgcggaagccggcgttcctgagcggcgagcagaagaaggcgatagtggacctcctcttcaagaccaacaggaaggtgaccgtgaagcaattaaaagaggactacttcaagaaaatagagtgcttcgactccgtggagatctcgggcgtggaggatcggttcaacgcctcactcggcacgtatcacgacctcctcaagatcattaaagacaaggacttcctcgacaacgaggagaacgaggacatcctcgaggacatcgtcctcaccctgaccctgttcgaggaccgcgaaatgatcgaggagaggctgaagacctacgcgcacctgttcgacgacaaggtcatgaaacagctcaagaggcgccgctacactggttggggaaggctgtcccgcaagctcattaatggcatcagggacaagcagagcggcaagaccatcctggacttcctcaagtccgacgggttcgccaaccgcaacttcatgcagctcattcacgacgactcgctcacgttcaaggaagacatccagaaggcacaggtgagcgggcagggtgactccctccacgaacacatcgccaacctggccggctcgccggccattaaaaagggcatcctgcagacggtcaaggtcgtcgacgagctcgtgaaggtgatgggccggcacaagcccgaaaatatcgtcatagagatggccagggagaaccagaccacccaaaaagggcagaagaactcgcgcgagcggatgaaacggatcgaggagggcattaaagagctcgggtcccagatcctgaaggagcaccccgtggaaaatacccagctccagaatgaaaagctctacctctactacctgcagaacggccgcgacatgtacgtggaccaggagctggacattaatcggctatcggactacgacgtcgacgccatcgtgccgcagtcgttcctcaaggacgatagcatcgacaacaaggtgctcacccggtcggataaaaatcggggcaagagcgacaacgtgcccagcgaggaggtcgtgaagaagatgaaaaactactggcgccagctcctcaacgcgaaactgatcacccagcgcaagttcgacaacctgacgaaggcggaacgcggtggcttgagcgaactcgataaggcgggcttcataaaaaggcagctggtcgagacgcgccagatcacgaagcatgtcgcccagatcctggacagccgcatgaatactaagtacgatgaaaacgacaagctgatccgggaggtgaaggtgatcacgctgaagtccaagctcgtgtcggacttccgcaaggacttccagttctacaaggtccgcgagatcaacaactaccaccacgcccacgacgcctacctgaatgcggtggtcgggaccgccctgatcaagaagtacccgaagctggagtcggagttcgtgtacggcgactacaaggtctacgacgtgcgcaaaatgatcgccaagtccgagcaggagatcggcaaggccacggcaaaatacttcttctactcgaacatcatgaacttcttcaagaccgagatcaccctcgcgaacggcgagatccgcaagcgcccgctcatcgaaaccaacggcgagacgggcgagatcgtctgggataagggccgggatttcgcgacggtccgcaaggtgctctccatgccgcaagtcaatatcgtgaaaaagacggaggtccagacgggcgggttcagcaaggagtccatcctcccgaagcgcaactccgacaagctcatcgcgaggaagaaggattgggacccgaaaaaatatggcggcttcgacagcccgaccgtcgcatacagcgtcctcgtcgtggcgaaggtggagaagggcaagtcaaagaagctcaagtccgtgaaggagctgctcgggatcacgattatggagcggtcctccttcgagaagaacccgatcgacttcctagaggccaagggatataaggaggtcaagaaggacctgattattaaactgccgaagtactcgctcttcgagctggaaaacggccgcaagaggatgctcgcctccgcaggcgagttgcagaagggcaacgagctcgccctcccgagcaaatacgtcaatttcctgtacctcgctagccactatgaaaagctcaagggcagcccggaggacaacgagcagaagcagctcttcgtggagcagcacaagcattacctggacgagatcatcgagcagatcagcgagttctcgaagcgggtgatcctcgccgacgcgaacctggacaaggtgctgtcggcatataacaagcaccgcgacaaaccaatacgcgagcaggccgaaaatatcatccacctcttcaccctcaccaacctcggcgctccggcagccttcaagtacttcgacaccacgattgaccggaagcggtacacgagcacgaaggaggtgctcgatgcgacgctgatccaccagagcatcacagggctctatgaaacacgcatcgacctgagccagctgggcggagac

SEQ ID NO: 49 - DNA, 23 bp, Zea mays
  source 1..23: mol_type = genomic DNA
ttccacagggagggagcaaacaa

SEQ ID NO: 50 - DNA, 23 bp, Zea mays
  source 1..23: mol_type = genomic DNA
cataatgaggatcgaggatgagg

SEQ ID NO: 51 - DNA, 49 bp, Cauliflower Mosaic Virus
  source 1..49: mol_type = genomic DNA
cgcaagacccttcctctatataaggaagttcatttcatttggagaggac

SEQ ID NO: 52 - DNA, 67 bp, Tobacco Mosaic Virus
  source 1..67: mol_type = genomic DNA
tatttttacaacaattaccaacaacaacaaacaacaaacaacattacaattactatttacaattaca

SEQ ID NO: 53 - DNA, 678 bp, synthetic construct
  source 1..678: mol_type = other DNA; note = synthetic construct
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtag

SEQ ID NO: 54 - DNA, 950 bp, Oryza sativa
  source 1..950: mol_type = genomic DNA
agctgctgctgttctagggttcacaagtctgcctatttgtcttccccaatggagctatggttgtctggtctggtccttggtcgtgtcccgtttcattgtgtactatttacctgtaatgtgtatccttaagtctggtttgatggtgtctgaaacgttttgctgtggtagagcagcatggaagaactataatgaataagtgatccctaatcattgtgtccaaattttgcttctgctatacccttttgtgctgtttcttatgttttgcttaaaaatttgatctgacaaacaaatttgtctaaattatgctcttgttctgactgtgtactgtacacatttgtattgctaccggtttggcacatagttttctctttgattgcagatcaaacccttctgattgcagattgcttggctatggctgatgtgctcactgttcttctttagcttgtatattgctgatggtggcttggctacggctgaaaaagttgctgctgcgccgacttggcaactgttgctccagtaatgctttgtcctctcttcctagccgccgacttggcacttggcagctgttgctcctgtaatgcgttgttatctcttatctcttgagatgtactatgattttgaggctgtttgacttgtgccagattgccagtgctgatgctatattatctcaggtttttatgctagtatatgttttactgtgtgctgcctgtgtcgagttacgtaatctattgaaccgctatgcgtgttgatgccctgttgttgcttgccagattgatgcccctgttgcttgcagcttgcaagctgtagtgaaatcctaaaattccaaaccaaatctattctgaacttttgaagatctgtgatgtccggaagaaaagattcaaacagataaacgggactctccagaatctataaaataaatgatcgacaaacgctttgctttggtaggcttc

SEQ ID NO: 55 - DNA, 13643 bp, Zea mays
  source 1..13643: mol_type = genomic DNA
tctaacgccgacaccgtcgacgagtaagaggtcgtggccggctgcccacacgtctgtgtgaacctcaccatcgacaccaccgtccgcctgcggcctacggcctcgaactccaacatcacattcctctacaactgcatgaagaacattaccctaccctctgtcatggaactgagtgggttcccacaacaacaagaagatagatgtaggtcgtacgtgctgtgggatggcgggatcacgggtgctcaggcgtacgggtatgggtgcgaggacttggtggtagcgtcggagctggatgtacacaaaaagggagaaggcgatccacctccggaacggatcgctccatcgggttgccgcatgacgggatcgagctgaactacgacactcactctaagcagtgggacctagggatgaaaacggtcggtaaacactaaagcaattaccgttttcatattttttttatcgaaaacaaaatcgaaaacggtaactccggaaatggaaacgatatcggtatttcggaaacatcgcaaacgaaagttcggtgcgaaaaatacacaagtaacggtcgaaatctaaaatacgatcgataaacatatcaaacttcataatacaacaaagttgacaaaagatcacaaagaccacaagttcacaattcatgatataacaagtttaaaatataacaagttcacaaggatcacaaatttataatataaaaaaattacaaagatcacaagttcacaatataacaagttcacagtataacaagttcataaagatcacaagttcacaaactcaaggttcacaattcacaatatccactcgatttagctgacatggcatgcttactcatgaaatattttttcctcaaataaagagtttttcacagactcgtaggaaaaccctaaaatctagtatgtggaaaagacagactgtcggctctatcattatatattactaatacataacatgtggacagaaaatggtggattcgttcgagagaccaaatcgttaatctcaactgccttccaacaccatctatcaaaaaaaatcttaaggcgtccaaaaaatacaaaaaataagtaatccttatctagagacgtacaggcgatgcgaaaaaaatcacatgctgaataattccgaaaaaaattccggaaaattctgagacataaatccggtaatttccgacaaaaaccggtaaccgaaggaaacagtcggtaaaacaccacgccgattccgataccgactctaatagaaatttccgaaaaccgatttcgttttcgaaaaatactgttaccggtgaatacaatcgaaaaatttcgaaatcggtttccggaataccaaaaaattgtgaaactgttttcatcactagtgggaccgatgtgatggctccgacggatggtgtggctaccagcgtgatgagacgcccaccggcgggatgacattcgcatgtttttgcaaacgcagcccgatcacatgtgcacaatcgttagtggcgcgttttaaaaaagacgtaactacgaaaaaaaatagcgacatgtttttagccagtgtcacaaacgtcatcttcgagcacttttgatgtagtgtatacaatctactagtcaaattaaaaagaataacatacacgaaatggttcataacttttatatagttaaacaataataaatttataaaaatccctaaacaaacaacaaaacgttggacacctccccgctatttatggcggatggcccgcccaattgacgtaatttccacatcgaagtcgaagaaccggtcgctgtgaccagtcaccccaacctccgtcacttgtctctcacgcgcgcgcaacatcgccgagaaaaccagaacagacctccggattggtctctcccccgttcacacgagcacattgcgatggctcctctgctcctgctgctcctcttccccgtccagctccccctcgcagtagccgacgccgtctccggtccctgcaccagagccacatgcgccggccaggacgtccattacccgttctggctcaagtcctccgcgcccgactgcgtctatcccggtgtcggccttgtggcccttgtctgcgagggcaactcgacgctgatcctccccttcaagtcccacagatacgtagtgctcagcatcgactacaagacgcgtaccgtgctggtctccgacgccgacatcgtccacgagtacgacgcggccggctgcccgcgcgtccgcgtgaacctcaccatcgacaccgcctggctgcggcccacggcctccgactccaacatcaccttcctctacaactgcaagaagaacatcaccctgccctccgccgtggaactgagcgggtgccagcagcagcagcaacaagacggcagcaggtcgtattcgtacgtgctgccggacggcggggtcacgggcgctgaggcgcaccagtacgggtgcgaggacttggtggtggcgccggtgctggacgtccacaggagggcgatcttgagggcgcctggcggcccgactctggagaacgggtcgctccgtcggttgctgcagggcgggttcgagctgaactacgacactcactccgagcagtgcgaccgatgcgaggcctccggcgggtggtgtggctaccagcgcgacgagacgcccgccggctggatgacgttcgcctgcttctgcgacggcgggccgacgaccacggcccgatgcggtgccggtatgtcttttttttttcttcgaacaaggtgtggcatgtgttctaccgtttcaactagtaaatgattacattgagctaggcagctagccacacattttcttgaatgattttctttgatgaacctgctgtttgcttttatgacgtgaacaacggggcctgcaagctgccacatacctagggagactagttcgtgaccttctttacacgtcttctctactcgccactgggagttgacgccgctcggtcgtcccactttgtgacgttcaaccagagtctagagatgtaattctctgcgaatacaggactagttggagctaacaacgcagcttgacgagggtgaacccagctccacctcgtctaccacgtcttctcctcgccatggctgctcacctaccacgcctccccgtcctcctcctcgtcctcctcgctgctcatgtcgtctccacctccgcccatgccgagcctcctcttccgagcccttacagcacctccgcccatggcgagcctcctcttccgagcacttacaacgtctccatgtgctcggaatcgttctggtgcggcggcgtcgaaatccgctacccgttctatcttgccaacgcaaccgccgactacagcgggagctactactcctgcggctacaccgacttgagcgtttcctgcaaactcgaggtcgaggggccgacgacgacatggacccctaccatccgtctcggcggcgacaactacaccgtcaagaacatcttgtacgactatcataccatctcactggcggacagcgatgtgctcggaggcggcgagtgccccgtcgtccaccacaacgtcagcttcgacgagacgtggctgcacaaccccagcgccttcgacaacctcaccttcttcttcggatgccactgggggccacgcgatacactgcctgaatttgccggcaacaacatcagctgcgccgggttcagtactccagctatcagcggtggaggctccttcgtgttcaagcctgaagatcttgacgaacatgcggagcaggagttggcttcacactgcgacgaggttttctccgtgccagtgagaagcgaggctctgcagcaggcgatcgtcagcaacctcagcctcggggacgggtacggcgagctgcttaggcaggggatcgagttggaatggaaacggacatcggaggatcagtgtggccagtgcgaggaatcgggctccggcggacggtgcgcctacagccagaagagagaattccttggctgcttgtgcagcggagggaaggcgggcaacccgttctgcaaaccatcaagtaaagtcctgaaccgagcctcccttatttttttttcattttttgcaatccaccagagagcacgcatcggttgcgtcagtatcttgcaacctcgtagctagccccgcagtgtcccctgtgtgcgagtaccgcgctgctccagcttgcctcctgctaacgcctaacggtgaatgcttcatgcttgacatgatctagctagtctacactttgcttggggtttgcctgggagctggaaattctggctcctgtttgcatcactcgacaaggacgctttcagacttgcgactctcgttctgcttttgcaccaaatccgtgtttttttcatttcgtgatcgagattaatctagcttagagatgacaatgggtatccgaaactcgaaacttgatggatttttactccattagggtataggtttgaatcaattttcatatttatggatttgttaataggcataaatatatatccaacaggtttatagatacgagtttgtttctacagtactcaaatccgtgaacacatgaggtttttaaacccgaccaaacctagtgcatattgtcattttattttataaacgaacaacaaaattgttatctctatttacttcctattttttatcgattggtgaatgtataagtagttggtgagaatgtttcttgcttgctattatagttttactagcgttatatatgttgtgggtggataacttagtgcaatgtcacttgattatacaacttattatttgtattcattctctctactaataatttttataccaaatcatgaactcggtgtttattatataaattttggaccataatctcattaatcatcacgatagttattgattatgagaaaaaacaagcatattggagataaaaccctcggctaacccgttaacccgatgggtacgggtttgaacaaaatttcaaacctattatgaatataagttttttaacaagtatagatatatttcacggatagagtttaagatgacaaaatccaacggatttgtatccattgccatctctatccggcggcccttacccggcggcccttaccgtgctccacgagcagaggtcgtatcgtccctcttcccgtgtcgcctgcttcgcgttgccgaacggagacgtttggtagcgttggccggctctagcagtcgggtcaactctttttgttgttgttttcgatgttgttggatttttgttccgtataagccatgttttagtaatttatttagtccagccgaatccgaagacgtgtttgctgggttggagactttggagttgctagtcatgatatgctttctactcggtttgatttcaacccagttaggctatatttaatactctagtatttatttcaatataaatggtttgaaacggattaaggtataaattagtttaatttatatatttaattcctctcaatccatatgtattgggctgaatactgaggtagtgtttggttgaagagccatatagaacggagccgttctgttccagttttgttgttgtttggttataaagtaactagaacggaatgacttcaattaaggaatattcttctcagatccagaaccattccgctctaaaaaatcaaccggacggagccgctccgtttccatcctgctcttacagtcacgctccgttccgttcactctgcaaccaaacaaaaaacagagccgctccgttccaaattaccaaacacagaacagagcggcttcattcctagaattaggaatggaacgactctgttctacttgactcctcaaccaaacactacctaagtatccaaacaagcccttatttaagatgcatttcctttacagttacacatgaccactattgtgtgggggcaggctgaacaagcccttatttaagatgcatttcctttagagttacacgtggccactattgtgggggggggggggggggggcaggccgatcctactcgtcagtgctcattcgagagcaaagataccgaaggagattagagagactaaaaactttttactatttaaaattagataataagacgatttaatcccgttccatatctttgctctaaacaaaccctcaatgatcatatatctcggaagatccggccggctgttctttatttatcaagtgatgactgctgaccgcttatagaatatatattttaaagcaaaatttcttctacagcagtaaaaggactagacgaaacaatgatgcatttctctaacaaaagaaagtagaattatcaagcggagagccaagaccaaaagccttacttctatgggcgtcaacaaatgataccgcgacggaaccatcccagcaggtctatactgtctgtcacgacccagcgagtaatcgtgtggctacgctattagacttaggattggatgaaatgctcggtttattaatgagccagctcgtgagttaagagtgtttggtttgatgaatgaagtaattcatcttcttttcactccccacttttttatttggtttgtgtaatagaatgagttgatccatcaccaccacattcatcataagctaataattagtatatacatgagtagtgagttgattccaccaaaattgatgaaatgaacttatgatgcatcatctcatgaagcatagagtgactccacaaaccaaacacaccataaatgatctattacataacgaaattatatgcatatcatttatcagggcgacgacagggggcatagggaggcaatatccccctaatgctccccaaattctatagggattgttagtttcttttagctaattctcatgtaaacaatataaaaaatgcttctaacagtccctcctaattataatttggtccaccctaatcttagatcctggcttcgtccgtgccatttacgtaacaattggtaaatatgttatacatgtgtggtatctatggcctatgaattgaactaatgattgatgaattgtgcttatgtgttaaattggtctatgcgaatataactatgggttaaacggatgaacatgtgtgttcattgttaattcatgagtgatgaattatgtataatttggtgttatattgatgtgttttgtgaaactatgtgtataattattattttctataattaaatttgtttgaaattaactagaaattgattattatatatatatatatatatatattgtttttctgctctagtctgcaagctaaacgagcaagctcaagctcgtaaacgatccgaaccgagctgactttgtggctcattaacttaacaagccgagttgggccaacttgttagcttaacgagtcagctcgaatacggacaagttgagccgagttggcatgatatccagccctaattaggcttgtaccagtgcaacatatccctctcgcctttgtcacgtccagacatgtcaatgggccccgattcccgcaaggaatttctctattaagggatgaggatgggaaagtttctccccccacagaaaaattctctcccgacggataagcggggacaacactccccatccccattccccgtggggacctattagacttacatatggtgatgttttcatgtaaaagttaatgataaaaataaacaattaccttgttgtcggcgtttcgaccccggagggtccctggaccgacgagtaaattgtcgctgcgtgtcccagcccagatgggtcgacgcgagacggaacacaagggggaaacaataaggggaatcgcggcctcgtgttgtcctgcgcccagggcggatgcgcttgcagtaaggggttacaagcgttcgcgagggagagagagagagagcctgtgcgccagcccgtcctcccgcgcggccaccttctcgtacgagggccctggacctttcttttatagatgtaaggagagggtccaggtgtacaacagggagcgtagcaatgtgctaacgtgtctagcagagggaagccagaatcctatgtacaggccgacgtgactgtcgaagaggttttggcgccctgttcatgtgatgtcgtggccgtcggaggagcgcttgagccctgtaggagcacagctgtcggagctgtcgggtccttgctgacgtctcattgcttccatagggagctgagaaccgccgtcgtcatggagcacgcggggtgccatcattacttgttttaccgggacgagccagatgggacgctggtcttgttcccagtagcctgaggtagctagaggtagggtaatgatgtgccctcctgcgacgtggtcggtccgagcccaaggtcgggcgaggcggaggctcctccgaggtcgaggctgagtccgagacctggggtcgggcgaggcggagaccgtcgtccgaggtcgaggttgagtccgagccctggggtcgggcgaggcagagtccatcgtccgaggtcgaggttgagtccgagccctggggtcgggcgaggcggagacagtcgtccgaggtcaaggttgagtccgagccctggggtcgggcgaggcggagttcgtcttccgaggtcgaggtggagtctgagccctagggtcaggcgaggcggagaccgtcgtccgaggtcgaggttgagtccgagctctggggtcgggcgaggtggagcttcctatggcgcctgaggccggacttggcggctgtcagcctcaacctgacgggtggcacagcagtcgaagcagcgcaggcggcgctgtttttctatcaggtcagccagtggaggggcgaagtgactgcggtcacttcggctctgtcgactgaagagcgtgcgtcaggataaggtgtcaggcgatccttgcattgaatgctcctgcgatccggtcggctggcgaggcgatcttggctaaggttgcttctccgcgaagcctgcctgagctgggcctcgggcgagtcggaggtgcgcccgttgcttgaggaggccctcgggcgaggcgtgaacctgcctgggcctgctgtttctgcccgaggctgggctcgggtgaggcgagatcgtgtcccttgagcggacagagctttgtcctgtgttgcgcccatcaggcctttgcagctttgtgctgatggtgtttaccagccgagtttaagagtcttgggggtacccctaattatggtccccgacacttgtcaagagatcactttttgtacaaatatattcattctgatgtacacatatttttttcttacatctaacaatgtgtataagtgagaatgttttatattaataacaaatgaagtatgtcctaattagacttctcacattgagcagcaacaaaacattttatgaattagtaaccattttacttactaaaataattactattgtatcttaatcattttgtgcaaaataaagaatgaaccaaattttgggtcaggtgtgggtcactcgcaggttgaaataaaaacccgcacccacactcgtgaaactttgggtcagatgcgggttacacccgcggattaaaatctctacccatacccacactcatcagatcgagtacccaaagattttaagtttgcgggttaaattgtcatccctcgacaccaacgtgttggaggatttggaagtttcgcgatgcgtacttaccaagtgtttggttctatgataaaagtttagcctgtgtcgcattagatgattgaatgtctattatgagtattaaatattgtctaattattaatcaaattacacaagtgaaggctaaacaacgagacaaatttattaagcctaattaatctatgattagcaaatgtttacagtagcaccatctagtagcaccatctgagcgaatcatgaactaattaggtttaataggttcgtcttaacgtttaattcttatctatgtaattagttttataagtaaattatatttaatacttctaattagcctccaaacattcgatataacatagactaaaatttagtcagtggtgctgctgctggctcttttccttcatccgacgtaatgttttcctcccaatttattgtgttgttttgtttgtgctttaaaactgacttgctctctgcttgctgtatcaaagtcgtgatgtgtgcttagcttattcccttccttgctagctgcccttagcatctatatagggatatttggttatagggactaaagtttagtttagtccgtgtccaattataaaactaattacataactgaatactaaaagacgagaaaattttattaaacttaattaatttatgattagcaaatgtttattgtaacatcacataagtaaattatagactaattttgtttaataggttcatcttatcatttagtcttcatctatgtaattagttttgtaattagactatatttactatttctaactagtatctaaacattaaatgtgacatggattaaaatttagttagagcaactccagtagttttctaaaagacttcctaaatcaataatttaggtagttaacatgaaaactattctccaacagttctctaaataaactttctaaatttaacaacttgtcatctaacctcattttctctctacatttggcaaccatttaataactccctaatcaaaaatgttgactgcattatatagtttttgtgacttattttttatgtggataaatacaaaataaaattacaacctatatttagagaactattggagaacccacttatttttatttcaaaagtcatttagcaacttcttaaatctgtaatttagaaagctaaaatttacataactattagagttgctcttagtgacgttaggcttttagagttgagttggtcgtagcttggtttagttacgtgtttgttctcttgcgtttatttaggacctctctataatgttttatcaccttcttaatacaaattaaaatacgcagctctcttgcgtattggaggcgtgtgttcctatattttttaggctcaattatgaatgaaattattctcagcgagcatataaccgttttcgaggtaaaatgaactaaaagcatatgggcctgcctgttcgcataagtacaccctccgtctaaaaaagaataaaaatatcatttcttgatgagtcaaaaaagttcaaatttaagaaaatatatgttacgacaccaatatttataatgtgtaataagtactgctgattaattttaaaataaaattttcataataaacctatttgaagatacaagtattggtactatttctaataaatctaatcaaactggtgttatatcttttgtaacaaatttgtgctttgtgtttctggttgacgtgaatcagcttaatcttgctgaaatctaacattgtcttttgttcgttggcatacaggatcaaaaaggaaagaagcatctattgttggtaagagcctatagtcaatacccatgttcatttcgtctaaaagagcagaagaaaagcatatgatgaattattgccatgtcatgtttaaaatacagaattctcaaaaacaaaaacaaaaaaaacttggaatccactaaccagtaaccactgatagcattgtagaaaatttcatcctccctttgggcaatacactgatgagtttacatgctgactagtggtgcatttgttctttgccaattgaatttttagaatgctttgcagctgaattcacttgtgatttttttttgtgatgcaggtgctgttgccgttgcattcctgtgtctagtcattctcacatgcttcttggcttgtagacatggttcgctgcccttcaaatcggagaacaaaccagggacaaggattgagtccttcctacagaagaacgagagtatacatccgaaaagatacacctacacggacgtgaaaagaatgacaaaatccttcgctgtgaagctaggccaaggtgggtttggtgctgtatacaaaggcagcctccacgatggccgacaggtagcagtcaagatgctcaaggacacccaaggtgacggcgaggaattcatgaacgaggtggctagcatcagcaggacttctcatgtcaacgtcgtgacacttctagggttttgcttgcaagggtcgaaaagagcactgatctacgagtacatgcccaatggttcgctcgaaaggtatgccttcaccggtgacatgaacagtgagaatttgctaacctgggaaaggctatttgacatagcaattggcacggccagagggctcgaatacctacaccggggatgcaacactcggatcgtgcattttgacatcaagccacacaacatcctgttagaccaggatttctgtcctaagatctctgactttggactggccaagctatgtctgaacaaagagagcgctatctccattgttggcgcaagagggacgatagggtatatcgccccggaggtctactcaaagcaatttggaacaatcagcagcaagtctgatgtctatagctatgggatgatggtccttgagatggttggagcaagggaaaggaatacaagcgcaagcgcagatagtgaccatagcagccaatatttccctcagtggatttatgaacatttggacgactattgtgttggtgcttccgagattaatggtgagaccacagagctcgtgaggaagatgatagttgtaggtctgtggtgcatacaagtgattccgactgatcgaccaacaatgacgagagtcgtcgagatgttggaagggagcacgagtaatctagagttgccacccagagttctcttgagctgacaaagcgtagatatttttcctatcaaatgttgcttccaggtcacacaaatgcaaaatatttgtggagacgagtgcctatttacctcatacactgtatctgtatgacaaaagtcccacgactcactggacgcggaaatgtcgcttgactacgccaattttctaaaaagattggcagcaattaatggaggcttatagcggtaactttggttcgcattaatcctaggactagggttgaatatcgatctaactcgacgcggcttggtcaagctcaagctagctccactcatctcattaaagaatccagctagaaaatcaacccaagtcgtttacgaaacgagtttgagctgactcgtttagatcgtaaatcacaacaaaaacaatatgcacatatatacaataatataatcaatactagttaattctagactagtttaacactagaaaagagtaatgatactcataatttcacatacaatgtcaatccaacaccaatttaacacacttcatcacttattagttcatccaaccaagtgtaggctttgatttactaacaaatggttgctcgttcgagctagcgagcttgcttgttaacaaactgagttgagatgctagcttaacttgtgacaaaattaaaacgagccgagtcgagtcaagttgagctcacgatgagtcgagcaagctcacaatccacgagtatttttttagtcctatctaagactaaagtttaatcctaaaactaaattttaatctctatttgtttggttctataaactaaacaggttcagaaaacataaaatacattatagaaaacctgaaatacccttctatacttaaggcatcactaagagagagcaataaataaagggtagagagaggaataaatctgctttattcccttttagctaccctttgagagagtaaacactaaaatgaaaggatccttgaggattttgatgttttggatgacaactaacacaattaaaggtctaattaggatgttaa

SEQ ID NO: 56 - DNA, 50 bp, Zea mays
  source 1..50: mol_type = genomic DNA
tcaaagttttctagctttgcatcatgtcttgaacaatgattgctcctcat

SEQ ID NO: 57 - DNA, 1000 bp, Zea mays
  source 1..1000: mol_type = genomic DNA
cctcgatcctcattatgcaccatggaccactatctaatccagtccgtggccttgtaggttacgacgccgatttcaggatccagatgtgattttcattagtctcacaaaggatgcactacctccgttcttcatttttgaactgaattgtgacaaataaaaaaaataaagggataggatgtaaatctaaaatttcagctgcaggcggcggagaccgtgcgtggcgcgcggcacaagcacaacctgaacgcacggagccaaggtctgtccctcccgccgaccgccgcgcgagcgctggttctgttccagccgcgatagatacacgccgtgtccatatcagatgcctccatggacatgtgacggggatcgtaccacttgtcatgtcctatcgatatgctagacgccatctatccgtgcatgctcctaacgaagggtgtctgttgaaggtgagttatggtgtctttcctcgatcctcgtacgagtacgaacgacacgcatgatcatcatcatgtaagcacgcacgcagccatcagctagccttgctgcaagccagtaagtaggatatgtactaggatcctcctgcctgccctgatgcttcttgcctcccccgttttttttttttaccaggtttcacgcttcagaaggcacacggctgacggctgcacaccagtgcattatatatacagatgcagtagtatgtgtagctaagtttggtattggaaaaccatggtatacacctagctatgacattaatagcatgcatggttttaatttcttcagccaactgaaactatggtattttttggtagtatataagtataattgaacaccaaactcatatatagaagcatgcaaactcacggcttgccaacgtgaacagcagccaacgccgctgtgaacagcaaccacgggaactaccaaaactcattcatattcagcacaacataaactataaaaactcaaccggcacacaaaaaaaaagtaccaggggaacagcagcaac

SEQ ID NO: 58 - DNA, 23 bp, Zea mays
  source 1..23: mol_type = genomic DNA
ttcacaagtctgcctatttgtct

SEQ ID NO: 59 - DNA, 500 bp, Zea mays
  source 1..500: mol_type = genomic DNA
ccggggacgacgacgcgcaggcatggagacacgcagacgtgccatcgcgcgctttggggaatgggaactagaggcgcggaccaacgtcgtacgccgaacgcactgatcggccgtccatcagcaccgtgtggtgctcgtgcaccgcggccggtggccggtgcgcgggtgggcgatggggctgccacaagggaacgacgccgaccgacggcatgcagacgacgcacatcatcacataacctgaaagctcgtggcatgcgtgatgcgcgcgtgtgttgactcttgcgctgttagaacctggctagctagcgcgcgagacggcgtctgtcgactcaggcaggagggagggatgagtcggggagatggggctttcggaatctctctcgtctttccaatggcggggccggcctgaccccgatcattgatcccactagcgcctgaattattgcccgaaagctaatgtgcgtgcggtgcgatgacccaagatcctcttcttcctcgat

SEQ ID NO: 60 - protein, 264 aa, Escherichia coli
MVEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGDDLVVTHGDACLPNIMVENGRFSGFIDCGRLGVADRYQDIALATRDIAEELGGEWADRFLVLYGIAAPDSQRIAFYRLLDEFF

SEQ ID NO: 61 - protein, 391 aa, Escherichia coli
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 62 - protein, 302 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

SEQ ID NO: 63 - protein, 710 aa, Zea mays
MATVNNWLAFSLSPQELPPSQTTDSTLISAATADHVSGDVCFNIPQDWSMRGSELSALVAEPKLEDFLGGISFSEQHHKANCNMIPSTSSTVCYASSGASTGYHHQLYHQPTSSALHFADSVMVASSAGVHDGGAMLSAAAANGVAGAASANGGGIGLSMIKNWLRSQPAPMQPRVAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGGRMSAWGTVVSAAAAAAASSNDNMAADVGHGGAQLFSVWNDT

SEQ ID NO: 64 - protein, 225 aa, Discosoma sp.
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFL

SEQ ID NO: 65 - protein, 414 aa, synthetic construct
MSPERRPVEIRPATAADMAAVCDIVNHYIETSTVNFRTEPQTPQEWIDDLERLQDRYPWLVAEVEGVVAGIAYAGPWKARNAYDWTVESTVYVSHRHQRLGLGSTLYTHLLKSMEAQGFKSVVAVIGLPNDPSVRLHEALGYTARGTLRAAGYKHGGWHDVGFWQRDFELPAPPRPVRPVTQMDPPVATMASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFL

SEQ ID NO: 66 - DNA, 70 bp, Zea mays
  source 1..70: mol_type = genomic DNA
cttcacaaggacggccacaatcaaagttttctagctttgcatcatgtcttgaacaatgattgctcctcat

SEQ ID NO: 67 - DNA, 175 bp, Zea mays
  source 1..175: mol_type = genomic DNA
gtgcggcgtcatgttgttcaccttaactacaatacaaacatggctaacaacaaaaaagaactaattaagcttcgatagggttcatgaagttccccccggcctaaacttcacaaggacggccacaatcaaagttttctagctttgcatcatgtcttgaacaatgattgctcctcat

(19) United States
(12) Patent Application Publication
(10) Pub. No.: US 2026/0015624 A1
(43) Pub. Date: Jan. 15, 2026

(54) GUIDE RNA TRAPPED GENOME EDITING

(71) Applicant: PIONEER HI-BRED INTERNATIONAL, INC., JOHNSTON, IA (US)

(72) Inventor: JOSHUA K. YOUNG, JOHNSTON, IA (US)

(73) Assignee: PIONEER HI-BRED INTERNATIONAL, INC., JOHNSTON, IA (US)

(21) Appl. No.: 18/881,874

(22) PCT Filed: Jul. 19, 2023
(86) PCT No.: PCT/US2023/070500

§ 371 Date: Jan. 7, 2025

Related U.S. Application Data
(60) Provisional application No. 63/369,550, filed on Jul. 27, 2022.

(51) Int. Cl. C12N 15/82; C12N 9/22; C12N 15/11

(52) U.S. Cl. CPC C12N 15/8213; C12N 9/226; C12N 15/11; C12N 15/8205; C12N 2310/20

(57) ABSTRACT

Methods and compositions are provided for driving expression of a coding sequence that has been integrated within the genome of a cell. A donor polynucleotide is integrated within the genome so that a non-functional promoter is located downstream of a genomic target sequence. This genomic target sequence can be bound by a site-specific promoter activation tool, wherein the site-specific promoter activation tool drives robust expression of a coding sequence that is operably linked to the non-functional promoter.

### INCORPORATION BY REFERENCE

The present application claims priority to the 371 national stage entry of International Application Number PCT/US2023/70500 filed on 19 Jul. 2023, which claims benefit of U.S. Provisional Patent Application Ser. No. 63/369,550 filed on Jul. 27, 2022 the disclosure of which is hereby incorporated by reference in its entirety.

### FIELD OF THE INVENTION

The present disclosure relates generally to the field of plant molecular biology, specifically the targeted modification of polynucleotides in plants.

### REFERENCE TO A SEQUENCE LISTING

The official copy of the sequence listing is submitted electronically via EFS-Web as an XML formatted sequence listing with a file named “9260-US-PSP.xml”, created on Jul. 17, 2023 with a size of 100 Kb, and is filed concurrently with the specification. The sequence listing contained in this XML formatted document is part of the specification and is herein incorporated by reference in its entirety.

### BACKGROUND

Standard methods for genome modification of a cell using site specific nucleases utilize traditional methodologies that may limit speed and efficiency. These methodologies negatively impact product development timelines. Currently, site specific nucleases are engineered to bind and cleave a genomic DNA resulting in a double strand break of the genomic DNA. The cell machinery repairs the resulting double strand break and in the presence of a donor polynucleotide can integrate the donor polynucleotide within the genomic DNA at the site of the double strand break. These methods are inefficient and require the screening of numerous events to identify and isolate an event that contains a genomic insertion of the donor polynucleotide at a specific site within the genome of a cell. Accordingly, there remains a need for the development of a faster, more efficient system to inserting a polynucleotide donor at a specific site within the genome of a cell quickly and efficiently.

### SUMMARY OF INVENTION

In some aspects, methods and compositions are provided for

### BRIEF DESCRIPTION OF THE DRAWINGS AND THE SEQUENCE LISTING

The disclosure can be more fully understood from the following detailed description and the accompanying drawings and Sequence Listing, which form a part of this application. The sequence descriptions and sequence listing attached hereto comply with the rules governing nucleotide and amino acid sequence disclosures in patent applications as set forth in 37 C.F.R. §§1.821 and 1.825. The sequence descriptions comprise the three letter codes for amino acids as defined in 37 C.F.R. §§1.821 and 1.825, which are incorporated herein by reference.

FIG. 1 provides a schematic that illustrates the gene expression cassettes for use with this disclosure. FIG. 1 contains the following genetic elements and the associated alphabetic label:

A: Zea mays Ubiquitin promoter (SEQ ID NO:9);

B: Zea mays Ubiquitin 5′ UTR (SEQ ID NO:10);

C: Zea mays Ubiquitin intron 1 (SEQ ID NO:11);

D: Zea mays optimized sequence encoding SV40 NLS (SEQ ID NO:12);

E: Zea mays optimized gene encoding including ST-LS1 Intron 2 (SEQ ID NO: 13) for an engineered variant of SpaCas12f1 (SEQ ID NOs: 14-15);

F: Zea mays optimized sequence encoding bipartite NLS (SEQ ID NO:16);

G: Zea mays optimized sequence encoding CBF1 promoter activation domain (SEQ ID NO:17);

H: Zea mays Ubiquitin terminator (SEQ ID NO:18); and,

I: Zea mays optimized spycas9 gene including ST-LS1 Intron 2 (SEQ ID NOs: 19).

FIG. 2 provides a schematic that illustrates the gene expression cassettes for use with this disclosure. FIG. 2 contains the following genetic elements and the associated alphabetic label:

A: Zea mays U6 promoter (including a 3′ G to promote transcription) (SEQ ID NO: 20);

B: DNA sequence encoding a SpyCas9 guide RNA variable targeting domain for target cleavage (SEQ ID NO:21) or promoter activation (SEQ ID NO:22-31);

C: DNA sequence encoding a SpyCas9 gRNA minus the variable targeting domain (SEQ ID NO:32);

D: Zea mays U6 terminator (SEQ ID NO:33);

E: DNA sequence encoding a SpaCas12f1 (SEQ ID NO:34) or AsuCas12f1 (SEQ ID NO: 35) gRNA minus the variable targeting domain;

F: DNA sequence encoding a SpaCas12f1 or AsuCas12f1 guide RNA variable targeting domain for target cleavage (SEQ ID NO:36) or promoter activation (SEQ ID NO:37-45); and,

G: DNA encoding a ribozyme (SEQ ID NO:46).

FIG. 3 provides a schematic that illustrates the additional gene expression cassettes for use with this disclosure. FIG. 3 contains the following genetic elements and the associated alphabetic label:

A: Zea mays Ubiquitin promoter (SEQ ID NO:9);

B: Zea mays Ubiquitin 5′ UTR (SEQ ID NO:10);

C: Zea mays Ubiquitin intron 1 (SEQ ID NO:11);

D: Zea mays optimized spacas12f1 gene including ST-LS1 Intron 2 (SEQ ID NOs: 14-15);

E: Zea mays optimized sequence encoding SV40 NLS (SEQ ID NO:12);

F: Zea mays Ubiquitin terminator (SEQ ID NO:18);

G: Zea mays optimized gene encoding a nuclease inactive or dead (d) engineered variant of AsuCas12f1 including ST-LS1 Intron 2 (SEQ ID NO:47);

H: Zea mays optimized sequence encoding bipartite NLS (SEQ ID NO:16);

I: Zea mays optimized sequence encoding CBF1 promoter activation domain (SEQ ID NO:17); and,

J: Zea mays optimized nuclease dead (d) spycas9 gene including ST-LS1 Intron 2 (SEQ ID NO:48).

FIG. 4 provides a schematic that illustrates the DNA repair templates for use with this disclosure. FIG. 4 contains the following genetic elements and the associated alphabetic label:

A: SpaCas12f1 or SpyCas9 DNA target cleavage site (SEQ ID NO:50);

B: Minimal 35S cauliflower mosaic virus promoter (SEQ ID NO:51);

C: Tobacco mosaic virus 5′ UTR (SEQ ID NO:52);

D: Sequence encoding DsRed visual selectable marker (SEQ ID NO:53);

E: T28 terminator (SEQ ID NO:54);

F: Trait gene expression cassette (CXE20) (SEQ ID NO:55);

G: 378 bp of Zea mays sequence flanking 5′ side of genomic SpyCas9-gRNA DNA target cleavage site (SEQ ID NO:56);

H: 419 bp of Zea mays sequence flanking 3′ side of genomic SpyCas9-gRNA DNA target cleavage site (SEQ ID NO:57);

I: 70 bp of Zea mays sequence flanking 5′ side of genomic SpyCas9-gRNA DNA target cleavage site (SEQ ID NO:66); and,

J: 175 bp of Zea mays sequence flanking 5′ side of genomic SpaCas12f1-gRNA DNA target cleavage site (SEQ ID NO:67).

FIG. 5 provides a schematic that illustrates the insertion of donor polynucleotide from a T-strand within the genomic DNA of a cell and PCR assays used in the detection of a said insertion. FIG. 5 contains the following genetic elements and the associated alphabetic label:

A: PCR amplicon for unique junction 1 generated by site-specific DNA insertion;

B: PCR amplicon for unique junction 2 generated by site-specific DNA insertion;

C: Long-PCR spanning the entire DNA insertion; and,

Primers are represented as arrows and qPCR probe as a short dashed line.

FIG. 6 provides a schematic that illustrates the insertion of donor polynucleotide

from a T-strand into a genome editing tool DNA cleavage site of a cell and the use of one or more nuclease inactive genome editing tools capable of activating a promoter to select for said insertion. The symbol of the line through the scissors shows the proximity of the nuclease inactive gene editing tool to the non-functional promoter. This nuclease inactive genome editing tool serves to activate the non-functional promoter to drive robust levels of expression of the selectable marker and is therefore referred to as a “promoter activating genome editing tool” (site-specific promoter activation complex). FIG. 6 contains the following genetic elements and the associated alphabetic label:

A: Cas-gRNA target cleavage site;

B: Minimal 35S cauliflower mosaic virus promoter;

C: Tobacco mosaic virus 5′ UTR;

D: Selectable marker;

E: T28 terminator;

F: Desirable trait gene expression cassette (SEQ ID NO:55);

G: 419 bp of Zea mays sequence flanking 3′ side of genomic SpyCas9-Grna DNA target cleavage site (SEQ ID NO:57);

H: 175 bp of Zea mays sequence flanking 5′ side of genomic SpaCas12f1-gRNA DNA target cleavage site (SEQ ID NO:67); and,

The scissors symbol represents a cleavage capable Cas-gRNA complex; and,

The symbol of a line through the scissors represents a promoter activating Cas-gRNA complex (site-specific promoter activation complex).

FIG. 7 shows how the directionality of a donor polynucleotide insertion into the genomic DNA of a cell can be modulated by the placement (positioning relative to the DNA cleavage site) of one or more nuclease inactive genome editing tools capable of activating a promoter. FIG. 7 contains the following genetic elements and the associated alphabetic label:

B: Minimal 35S cauliflower mosaic virus promoter;

C: Tobacco mosaic virus 5′ UTR;

D: Selectable marker;

E: T28 terminator;

F: Desirable trait gene expression cassette (SEQ ID NO:55); and,

The symbol of a line through the scissors represents a promoter activating Cas-gRNA complex (site-specific promoter activation complex).

FIG. 8 depicts the scarless excision of the selectable marker from the genomic DNA. FIG. 8 contains the following genetic elements and the associated alphabetic label:

B: Minimal 35S cauliflower mosaic virus promoter;

C: Tobacco mosaic virus 5′ UTR;

D: Selectable marker;

E: T28 terminator;

F: Desirable trait gene expression cassette (SEQ ID NO:55);

G: Additional SpaCas12f1-gRNA target cleavage site for scarless excision of selectable marker expression cassette (SEQ ID NO:58);

H: Zea mays sequence flanking 5′ side of genomic SpaCas12f1-gRNA DNA target cleavage site (SEQ ID NO:59); and,

The scissors symbol represents a cleavage capable Cas-gRNA complex.

FIG. 9 shows the frequency of plants recovered that contain a targeted insertion as detected using PCR across the unique junctions (“Junction 1” and “Junction 2”) resulting from DNA insertion into the cleaved DNA target site. Canonical methods don't use promoter activation of a selectable marker (“No Promoter Activation”). The methods described herein utilize “Promoter Activation”.

FIG. 10 provides an aspect of the method, wherein a gene expression cassette containing the non-functional promoter operably linked to a selectable marker (SM) is first inserted at the boundary of a chromosomal region destined for crossover. A region 5′ of the non-functional promoter (MSNP) is targeted for Cas-gRNA cleavage (as shown with the symbol of the scissors). The resulting double strand break causes crossing-over of the non-sister chromatids from a first homologous chromosome to a second homologous chromosome. After the crossing over of the non-sister chromatids, the non-functional promoter operably linked to a selectable marker (SM) is located downstream of the genomic sequence that can be bound by the promoter activating Cas-gRNA complex. As such, the cross-over event results in robust expression of a selectable marker so that the cells containing this DNA repair outcome can be selected for. FIG. 10 contains the following genetic elements and the associated alphabetic label:

A: Cas-gRNA target cleavage site 5′ of non-functional promoter;

B: Selectable marker expression cassette with non-functional promoter;

The scissors symbol represents a cleavage capable Cas-gRNA complex; and,

The symbol of a line through the scissors represents a promoter activating Cas-gRNA complex (site-specific promoter activation complex).

FIG. 11 depicts an aspect of the method, wherein a gene expression cassette containing the non-functional promoter operably linked to a selectable marker (SM) is first inserted within a chromosome so that it is inverted with regards to the location of the genomic sequence that can be bound by the promoter activating Cas-gRNA complex. Double strand breaks at each end of the chromosomal region to be inverted release it from the genomic DNA. A DNA repair event that results in an inversion of the selectable marker cassette and associated chromosomal DNA brings the non-functional promoter operably linked to a selectable marker (SM) in proximity with the one or more promoter activating Cas-gRNA complexes. As such, the inversion event results in robust expression of the selectable marker so that cells with this DNA repair outcome can be selected for. FIG. 11 contains the following genetic elements and the associated alphabetic label:

A: Cas-gRNA cleavage target site 5′ of non-functional promoter;

B: Selectable marker expression cassette with non-functional promoter;

C: Cas-gRNA cleavage target site at distal boundary of region of interest;

The scissors symbol represents a cleavage capable Cas-gRNA complex; and,

The symbol of a line through the scissors represents a promoter activating Cas-gRNA complex (site-specific promoter activation complex).

FIG. 12 illustrates an aspect of the method, wherein a gene expression cassette containing the non-functional promoter operably linked to a selectable marker (SM) is first inserted in a first chromosome (for example “Chromosome 1” of FIG. 12). A region on a second chromosome (for example “Chromosome 2” of FIG. 12) and the gene expression cassette of the first chromosome is targeted for Cas-gRNA cleavage (as shown with the symbol of the scissors). The resulting double strand break releases the gene expression cassette from chromosome 1 so that this gene expression cassette can be inserted within chromosome 2. After the insertion of the gene expression cassette within chromosome 2, the non-functional promoter operably linked to a selectable marker (SM) is located downstream of the genomic sequence that can be bound by the promoter activating Cas-gRNA complex. As such, the cross-over event results in robust expression of a selectable marker so that the cells containing this DNA repair outcome can be selected for. FIG. 12 contains the following genetic elements and the associated alphabetic label:

A: Cas-gRNA cleavage target site 5′ of non-functional promoter;

B: Selectable marker expression cassette with non-functional promoter;

C: Cas-gRNA cleavage target site at distal boundary of region of interest;

D: Cas-gRNA cleavage target site at new chromosomal location;

The scissors symbol represents a cleavage capable Cas-gRNA complex; and,

The symbol of a line through the scissors represents a promoter activating Cas-gRNA complex (site-specific promoter activation complex).

### DETAILED DESCRIPTION

Various compositions and methods for efficiently inserting a polynucleotide donor at a specific site within the genome of a cell, for example a plant cell, are provided. In an aspect, a gene expression cassette is designed containing a non-functional promoter to drive expression of a coding sequence. Upon insertion of the gene expression cassette into the desired genomic location a site-specific promoter activation complex (for a non-limiting example; a promoter activating Cas and gRNA complex) binds to the regions flanking the genomic insertion site and is used to drive the non-functional promoter to robustly express the coding sequence. Various applications of this method can be utilized for different genomic configurations. For example the methodology can utilize genomic crossovers, inversions, and relocations to position the non-functional promoter within proximity of a genomic region that is bound by the site-specific promoter activation complex. Once the site-specific promoter activation complex is in proximity to the non-functional promoter the expression of a selectable marker is driven at robust levels to allow for marker-based selection. These novel methods and compositions provide improved efficiency of donor mediated insertion within a genome, genome editing, improved percentage of regenerated transformed cells, as for example plant cells, lower attrition rate of the target cells/organisms, removal of yield drag, and reduced integration of unwanted DNA.

### Definitions

Terms used in the claims and specification are defined as set forth below unless otherwise specified. It must be noted that, as used in the specification and the appended claims, the singular forms “a,” “an” and “the” include plural referents unless the context clearly dictates otherwise.

The term “genome” refers to the entire complement of genetic material (genes and non-coding sequences) that is present in each cell of an organism, or virus or organelle; and/or a complete set of chromosomes inherited as a (haploid) unit from one parent. The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

The terms “provided (to)” and “introduced (into)” are used interchangeably herein. In another aspect, it is meant that a particular composition becomes functionally associated with a cell or other molecule. In one aspect, it is meant that a particular composition is taken up by the cell into its interior.

By the term “endogenous” it is meant a sequence or other molecule that naturally occurs in a cell or organism. In one aspect, an endogenous polynucleotide is normally found in the genome of a cell; that is, not heterologous.

The term “heterologous” refers to the difference between the original environment, location, or composition of a particular polynucleotide or polypeptide sequence and its current environment, location, or composition. Non-limiting examples include differences in taxonomic derivation (e.g., a polynucleotide sequence obtained from Zea mays would be heterologous if inserted into the genome of an Oryza sativa plant, or of a different variety or cultivar of Zea mays; or a polynucleotide obtained from a bacterium was introduced into a cell of a plant), or sequence (e.g., a polynucleotide sequence obtained from Zea mays, isolated, modified, and re-introduced into a maize plant). As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more compositions, such as those provided herein, may be entirely synthetic.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single-or double-stranded, optionally comprising synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The relationship between two or more polynucleotides or polypeptides may be determined. Polynucleotide and polypeptide sequences, fragments thereof, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment.

Sequence relationships may be defined by their composition comparisons, or by their ability to hybridize, or by their ability to engage in homologous recombination.

Methods of alignment of sequences for comparison are well known in the art. Thus, the determination of percent sequence identity between any two sequences can be accomplished using a mathematical algorithm. Sequence alignments and percent identity or similarity calculations may be determined using a variety of comparison methods designed to detect homologous sequences including, but not limited to, the MegAlign program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

“Sequence identity” or “identity” in the context of nucleic acid or polypeptide sequences refers to the nucleic acid bases or amino acid residues in two sequences that are the same when aligned for maximum correspondence over a specified comparison window. The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%. These identities can be determined using any of the programs described herein.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign program of the LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign v6.1 program of the LASERGENE bioinformatics computing suite (DNASTAR Inc., Madison, WI). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program. Unless otherwise stated, sequence identity/similarity values provided herein refer to the value obtained using GAP Version 10 (GCG, Accelrys, San Diego, CA) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases. “BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. As used herein, “percent sequence identity” means the value determined by comparing two aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percent sequence identity.

It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any integer percentage from 50% to 100%. Indeed, any integer amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, an “isolated” polynucleotide or polypeptide, or biologically active portion thereof, is substantially or essentially free from components that normally accompany or interact with the polynucleotide or polypeptide as found in its naturally occurring environment. Thus, an isolated or purified polynucleotide or polypeptide is substantially free of other cellular material or culture media components when produced by recombinant techniques, or substantially free of chemical precursors or other molecules when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein. When the polypeptide of the invention or biologically active portion thereof is recombinantly produced, optimally culture medium represents less than about 30%, 20%, 10%, 5%, or 1% (by dry weight) of chemical precursors or non-protein-of-interest molecules.

As used herein, polynucleotide or polypeptide is “recombinant” when it is artificial or engineered, or derived from an artificial or engineered protein or nucleic acid. For example, a polynucleotide that is inserted into a vector or any other heterologous location, e.g., in a genome of another organism, such that it is not associated with nucleotide sequences that normally flank the polynucleotide as it is found in nature is a recombinant polynucleotide. A polypeptide expressed in vitro or in vivo from a recombinant polynucleotide is an example of a recombinant polypeptide. Likewise, a polynucleotide sequence that does not appear in nature, for example, a variant of a naturally occurring gene, is recombinant.

The terms “recombinant polynucleotide”, “recombinant nucleotide”, “recombinant DNA” and “recombinant DNA construct” are used interchangeably herein. A recombinant construct comprises an artificial or heterologous combination of nucleic acid sequences, e.g., regulatory and coding sequences that are not found together in nature. For example, a transfer cassette can comprise restriction sites and a heterologous polynucleotide of interest. In other embodiments, a recombinant construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments provided herein. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

“Open reading frame” is abbreviated ORF.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in its natural endogenous location with its own regulatory sequences.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

The term “homologous” in the context of a pair of homologous chromosomes refers to a pair of chromosomes from an individual that are similar in length, gene position and centromere location, and that line up and synapse during meiosis. In an individual, one chromosome of a pair of homologous chromosomes comes from the mother of the individual (i.e., is “maternally-derived”), whereas the other chromosomes of the pair comes from the father (i.e., is “paternally-derived”). In the context of genes, the term “homologous” refers to a pair of genes where each gene resides within each homologous chromosome at the same position and has the same function. [0039] The term “homologous recombination” refers to a reciprocal exchange at corresponding positions between between homologous chromosomes, such as between non-sister chromatids of homologous chromosomes during meiosis. Homologous recombination can also occur in somatic cells during mitosis (somatic crossing over).

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” or gene that has been “mutagenized” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated plant is a plant comprising a mutated gene.

As used herein, the term “morphogenic gene” means a gene that when ectopically expressed stimulates formation of a somatically-derived structure that can produce a plant. More precisely, ectopic expression of the morphogenic gene stimulates the de novo formation of a somatic embryo or an organogenic structure, such as a shoot meristem, that can produce a plant. This stimulated de novo formation occurs either in the cell in which the morphogenic gene is expressed, or in a neighboring cell. A morphogenic gene can be a transcription factor that regulates expression of other genes, or a gene that influences hormone levels in a plant tissue, both of which can stimulate morphogenic changes. As used herein, the term “morphogenic factor” means a morphogenic gene and/or the protein expressed by a morphogenic gene.

A “promoter” is a region of DNA involved in recognition and binding of RNA polymerase and other proteins to initiate transcription. The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225 236).

As used herein, “minimal promoter” or substantially similar term refers to a promoter element, particularly a TATA element, that is inactive or that has reduced and lower levels of promoter activity in the absence of upstream activation. In the presence of a suitable transcription factor, the minimal promoter functions to permit transcription. Typically the minimal promoter is the minimal stretch of contiguous DNA sequence that is sufficient to direct accurate initiation of transcription by the RNA polymerase II machinery (for review see: Struhl, 1987, Cell 49:295-297; Smale, 1994, In Transcription: Mechanisms and Regulation (eds R. C. Conaway and J. W. Conaway), pp 63-81/Raven Press, Ltd., New York; Smale, 1997, Biochim. Biophys. Acta 1351:73-88; Smale et al., 1998, Cold Spring Harb. Symp. Quant. Biol. 58:21-31; Smale, 2001, Genes & Dev. 15:2503-2508; Weis and Reinberg, 1992, FASEB J. 6:3300-3309; Burke et al., 1998, Cold Spring Harb. Symp. Quant. Biol 63:75-82). There are several sequence motifs, including the TATA box, initiator (Inr), TFIIB recognition element (BRE) and downstream promoter element (DPE), that are commonly found in the minimal promoters. Not all of these elements, however, occur in all promoters. That is, there are no universal minimal promoter. In some instances the minimal promoter may be referenced as a “core promoter”.

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated yet has an effect on cellular processes. The terms “complement” and “reverse complement” are used interchangeably herein with respect to mRNA transcripts, and are meant to define the antisense RNA of the message.

The terms “5′-cap” and “7-methylguanylate (m7G) cap” are used interchangeably herein. A 7-methylguanylate residue is located on the 5′ terminus of messenger RNA (mRNA) in eukaryotes. RNA polymerase II (Pol II) transcribes mRNA in eukaryotes. Messenger RNA capping occurs generally as follows: The most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase.

The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export. One or more RNA components herein are uncapped.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

The term “expression”, as used herein, refers to the production of a functional end-product (e.g., an mRNA, guide RNA, or a protein) in either precursor or mature form.

By “domain” it is meant a contiguous stretch of nucleotides (that can be RNA, DNA, and/or RNA-DNA-combination sequence) or amino acids.

As used herein, “expression” at a “higher level” refers to enhancement of transcription or translation by binding of site-specific promoter activation complex to specific site on DNA or mRNA. Preferably, “expression” at a “higher level” includes a significant change in transcription or translation level of at least 1% increase.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

The term “fragment” refers to a contiguous set of polynucleotides or polypeptides. In one embodiment, a fragment is 2, 3, 4, 5, 6, 7 8, 9, 10, 11, 12, 13, 14, 15, 16, 17, 18, 19, 20, or greater than 20 contiguous polynucleotides. In one embodiment, a fragment is 2, 3, 4, 5, 6, 7 8, 9, 10, 11, 12, 13, 14, 15, 16, 17, 18, 19, 20, or greater than 20 contiguous polypeptides. A fragment may or may not exhibit the function of a sequence sharing some percent identity over the length of said fragment.

The terms “fragment that is functionally equivalent”, “functional fragment”, and “functionally equivalent fragment” are used interchangeably herein. These terms refer to a portion or subsequence of a nucleic acid fragment or polypeptide that displays the same activity or function as the longer sequence from which it derives. In one example, the fragment retains the ability to alter gene expression, create a double strand nick or break, or produce a certain phenotype whether or not the fragment encodes the whole protein as found in nature. In some aspects, part of the activity is retained. In some aspects, all of the activity is retained.

The terms “variant that is functionally equivalent”, “functional variant”, and “functionally equivalent variant” are used interchangeably herein. These terms refer to a nucleic acid fragment or polypeptide that displays the same activity or function as the source sequence from which it derives, but differs from the source sequence by at least one nucleotide or amino acid. In one example, the variant retains the ability to alter gene expression, create a double strand nick or break, or produce a certain phenotype. In some aspects, part of the activity is retained. In some aspects, all of the activity is retained.

A functional fragment or functional variant shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native source polynucleotide or polypeptide, and retains at least partial activity.

“Modified”, “edited”, or “altered, with respect to a polynucleotide or target sequence, refers to a nucleotide sequence that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) association of another molecule or atom via covalent, ionic, or hydrogen bonding, or (v) any combination of (i)-(iv).

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

A “mature” protein refers to a post-translationally processed polypeptide (i.e., one from which any pre-or propeptides present in the primary translation product have been removed).

“Precursor” protein refers to the primary product of translation of mRNA (i.e., with pre-and propeptides still present). Pre-and propeptides may be but are not limited to intracellular localization signals.

An “optimized” polynucleotide is a sequence that has been optimized for improved expression in a particular heterologous host cell.

A “codon-modified gene” or “codon-preferred gene” or “codon-optimized gene” is a gene having its frequency of codon usage designed to mimic the frequency of preferred codon usage of the host cell.

A “plant-optimized nucleotide sequence” is a nucleotide sequence that has been optimized for expression in plants, particularly for increased expression in plants. A plant-optimized nucleotide sequence includes a codon-optimized gene. A plant-optimized nucleotide sequence can be synthesized by modifying a nucleotide sequence encoding a protein such as, for example, a Cas endonuclease as disclosed herein, using one or more plant-preferred codons for improved expression. See, for example, Campbell and Gowri (1990) Plant Physiol. 92:1-11 for a discussion of host-preferred codon usage.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single-or double-stranded DNA or RNA, derived from any source, in which a number of nucleotide sequences have been joined or recombined into a unique construction which is capable of introducing a polynucleotide of interest into a cell. “Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

A “polynucleotide of interest” includes any nucleotide sequence encoding a protein or polypeptide that improves desirability of an organism, for example, animals or plants. Polynucleotides of interest: include, but are not limited to, polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

As used herein, a “genomic region of interest” is a segment of a chromosome in the genome of a plant that is desirable for introducing a double-strand break, a polynucleotide of interest, or a trait of interest. The genomic region of interest can include, for example, one or more polynucleotides of interest. Generally, a genomic region of interest of the present invention comprises a segment of chromosome that is 0-15 centimorgan (cM).

The terms “knock-out”, “gene knock-out” and “genetic knock-out” are used interchangeably herein. A knock-out represents a DNA sequence of a cell that has been rendered partially or completely inoperative by targeting with a DSB agent; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a DSB agemt (for example by homologous recombination (HR), wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

“Introducing” is intended to mean presenting to a target, such as a cell or organism, a polynucleotide or polypeptide or polynucleotide-protein complex, in such a manner that the component(s) gains access to the interior of a cell of the organism or to the cell itself.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

As used herein, the terms “target site”, “target sequence”, and “target polynucleotide” are used interchangeably herein and refer to a polynucleotide sequence in the genome of a plant cell or yeast cell that comprises a recognition site for a double-strand-break-inducing agent.

A “target cell” is a cell that comprises a target sequence and is the object for receipt of a particular double-strand-break-inducing agent.

A “break-inducing agent” is a composition that creates a cleavage in at least one strand of a polynucleotide. In some aspect, a break-inducing agent may be capable of, or have its activity altered such that it is capable of, creating a break in only one strand of a polynucleotide. Producing a single-strand-break in a double-stranded target sequence may be referred to herein as “nicking” the target sequence.

The term “double-strand-break-inducing agent”, or equivalently “double-strand-break agent” or “DSB agent”, as used herein refers to any composition which produces a double-strand break in a target polynucleotide sequence; that is, creates a break in both strands of a double stranded polynucleotide. Examples of a DSB agent include, but are not limited to: meganucleases, TAL effector nucleases, Argonautes, Zinc Finger nucleases, and Cas endonucleases (either individually or as part of a ribonucleoprotein complex). Producing the double-strand break in a target sequence may be referred to herein as “cutting” or “cleaving” the target sequence. In some aspects, the DSB agent is a nuclease. In some aspects, the DSB agent is an endonuclease. An “endonuclease” refers to an enzyme that cleaves the phosphodiester bond within a polynucleotide chain. In some embodiments, the double-strand break results in a “blunt” end of a double-stranded polynucleotide, wherein both strands are cut directly across from each other with no nucleotide overhang generated. A “blunt” end cut of a double-stranded polynucleotide is created when a first cleavage of the first stand polynucleotide backbone occurs between a first set of two nucleotides on one strand, and a second cleavage of the second strand polynucleotide backbone occurs between a second set of two nucleotides on the opposite strand, wherein each of the two nucleotides of the first set are hydrogen bonded to one of the two nucleotides of the second set, resulting in cut strands with no nucleotide on the cleaved end that is not hydrogen bonded to another nucleotide on the opposite strand. In some embodiments, the double-strand break results in a “sticky” end of a double-stranded polynucleotide, wherein cuts are made between nucleotides of dissimilar relative positions on each of the two strands, resulting in a polynucleotide overhang of one strand compared to the other. A “sticky” end cut of a double-stranded polynucleotide is created when a first cleavage of the first strand polynucleotide backbone occurs between a first set of two nucleotides on one strand, and a second cleavage of the second strand polynucleotide backbone occurs between a second set of two nucleotides on the opposite strand, wherein no more than one nucleotide of the first set is hydrogen bonded to one of the nucleotides of the second set on the opposite strand, resulting in an “overhang” of at least one polynucleotide on one of the two strands wherein the lengths of the two resulting cut strands are not identical. In some embodiments, the DSB agent comprises more than one type of molecule. In one non-limiting example, the DSB agent comprises an endonuclease protein and a polynucleotide, for example a Cas endonuclease and a guide RNA. In some aspects, the DSB agent is a fusion protein comprising a plurality of polypeptides. In one non-limiting example, the DSB agent is a Cas endonuclease with a deactivated nuclease domain, and another polypeptide with nuclease activity.

As used herein, the term “recognition site” refers to a polynucleotide sequence to which a double-strand-break-inducing agent is capable of alignment, and may optionally contact, bind, and/or effect a double-strand break. The terms “recognition site” and “recognition sequence” are used interchangeably herein. The recognition site can be an endogenous site in a host (such as a yeast, animal, or plant) genome, or alternatively, the recognition site can be heterologous to the host (yeast, animal, or plant) and thereby not be naturally occurring in the genome, or the recognition site can be found in a heterologous genomic location compared to where it occurs in nature. The length and the composition of a recognition site can be characteristic of, and may be specific to, a particular double-strand-break-inducing agent. The cleavage site of a DSB agent may be the same or different than the recognition site, and may be the same or different than the binding site.

As used herein, the term “endogenous recognition (or binding or cleavage) site” refers to a double-strand-break-inducing agent recognition (or binding or cleavage) site that is endogenous or native to the genome of a host (such as a plant, animal, or yeast) and is located at the endogenous or native position of that recognition (or binding or cleavage) site in the genome of the host (such as a plant, animal, or yeast). The length of the recognition (or binding or cleavage) site can vary, and includes, for example, recognition (or binding or cleavage) sites that are at least 1, 2, 3, 4, 5, 6, 7, 8, 9, 10, 11, 12, 13, 14, 15, 16, 17. 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more than 70 nucleotides in length. The composition of the recognition (or binding or cleavage) site can vary, and includes, for example, a plurality of specific nucleotides whose compositions are recognized by the DSB agent. In some aspects, the plurality of specific nucleotides is contiguous in the primary sequence. In some aspects, the plurality of specific nucleotides is non-contiguous in the primary sequence. It is further possible that the recognition site could be palindromic, that is, the sequence on one strand reads the same in the opposite direction on the complementary strand. The binding and/or nick/cleavage site could be within the recognition sequence or the binding and/or nick/cleavage site could be outside of the recognition sequence. In another variation, the DSB cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs.

As used herein, the term “target recognition site” refers to the polynucleotide sequence to which a double-strand-break-inducing agent is capable of aligning perfectly (i.e., zero nucleotide mismatches, gaps, or insertions), and in some aspects, induces a double-strand break.

As used herein, the term “target binding site” refers to the polynucleotide sequence at which the double-strand-break-inducing agent is capable of forming a functional association, and to which it forms bonds with complementary nucleotides of the target polynucleotide strand, with perfect alignment (i.e., zero nucleotide mismatches, gaps, or insertions).

As used herein, the term “target cleavage site” refers to the polynucleotide sequence at which a double-strand-break-inducing agent is capable of producing a double-strand break, with perfect alignment (i.e., zero nucleotide mismatches, gaps, or insertions).

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327:167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes but is not limited to: the novel Cas-delta protein disclosed herein, a Cas9 protein, a Cpf1 (Cas12) protein, a C2c1 protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence. A Cas endonuclease described herein comprises one or more nuclease domains. The Cas-delta endonucleases of the disclosure may include those having RuvC or RuvC-like nuclease domains. A Cas protein is further defined as a functional fragment or functional variant of a native Cas protein, or a protein that shares at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity.

The terms “cascade” and “cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready Cascade”, “crCascade”, “cleavage-ready Cascade complex”, “crCascade complex”, “cleavage-ready Cascade system”, “CRC” and “crCascade system”, are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, optionally bind to, and optionally nick or cleave (introduce a single or double-strand break) the DNA target site.

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example US20150059010A1, published 26 Feb. 2015), or any combination thereof.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327:167-170; Makarova et al. 2015, Nature Reviews Microbiology Vol. 13:1-15; Zetsche et al., 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant element” is synonymous to a “portion” of a plant, and refers to any part of the plant, and can include distinct tissues and/or organs, and may be used interchangeably with the term “tissue” throughout. Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

“Progeny” comprises any subsequent generation of an organism, produced via sexual or asexual reproduction.

As used herein, the term “plant part” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

The term “isoline” is a comparative term, and references organisms that are genetically identical, but differ in treatment. In one example, two genetically identical maize plant embryos may be separated into two different groups, one receiving a treatment (such as the introduction of a CRISPR-Cas effector endonuclease) and one control that does not receive such treatment. Any phenotypic differences between the two groups may thus be attributed solely to the treatment and not to any inherency of the plant's endogenous genetic makeup.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein.

“Agronomic trait potential” is intended to mean a capability of a plant element for exhibiting a phenotype, preferably an improved agronomic trait, at some point during its life cycle, or conveying said phenotype to another plant element with which it is associated in the same plant.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%), at least about 400% or more higher than the control.

The meaning of abbreviations is as follows: “sec” means second(s), “min” means minute(s), “h” means hour(s), “d” means day(s), “uL” means microliter(s), “mL” means milliliter(s), “L” means liter(s), “uM” means micromolar, “mM” means millimolar, “M” means molar, “mmol” means millimole(s), “umole” or “umole” mean micromole(s), “g” means gram(s), “ug” or “ug” means microgram(s), “ng” means nanogram(s), “U” means unit(s), “bp” means base pair(s) and “kb” means kilobase(s).

### Compositions and Methods for Modifying the Genome of a Target Cell

In some aspects, methods and compositions are provided for engineering a site-specific promoter activation complex within proximity of a non-functional promoter. In certain embodiments of this aspect, the application of methods and compositions for engineering a site-specific promoter activation complex within proximity of a non-functional promoter are utilized to edit a genome. For example, a donor polynucleotide can be inserted within the genome and selected using the methods and compositions as provided herein.

In other aspects the engineering a site-specific promoter activation complex within proximity of a non-functional promoter results in higher levels of expression of the non-functional promoter. In certain embodiments the non-functional promoter comprises any polynucleotide sequence upstream of a coding sequence. The length and composition of this polynucleotide sequence include a non-functional promoter comprising a polynucleotide sequence of at least 10,000 bp, a non-functional promoter comprising a polynucleotide sequence of at least 9,000 bp, a non-functional promoter comprising a polynucleotide sequence of at least 8,000 bp, a non-functional promoter comprising a polynucleotide sequence of at least 7,000 bp, a non-functional promoter comprising a polynucleotide sequence of at least 6,000 bp, a non-functional promoter comprising a polynucleotide sequence of at least 5,000 bp, a non-functional promoter comprising a polynucleotide sequence of at least 4,000 bp, a non-functional promoter comprising a polynucleotide sequence of at least 3,000 bp, a non-functional promoter comprising a polynucleotide sequence of at least 2,000 bp, a non-functional promoter comprising a polynucleotide sequence of at least 1,000 bp, a non-functional promoter comprising a polynucleotide sequence of at least 900 bp, a non-functional promoter comprising a polynucleotide sequence of at least 800 bp, a non-functional promoter comprising a polynucleotide sequence of at least 700 bp, a non-functional promoter comprising a polynucleotide sequence of at least 600 bp, a non-functional promoter comprising a polynucleotide sequence of at least 500 bp, a non-functional promoter comprising a polynucleotide sequence of at least 400 bp, a non-functional promoter comprising a polynucleotide sequence of at least 300 bp, a non-functional promoter comprising a polynucleotide sequence of at least 200 bp, a non-functional promoter comprising a polynucleotide sequence of at least 100 bp, a non-functional promoter comprising a polynucleotide sequence of at least 50 bp, a non-functional promoter comprising a polynucleotide sequence of at least 40 bp, a non-functional promoter comprising a polynucleotide sequence of at least 30 bp, a non-functional promoter comprising a polynucleotide sequence of at least 20 bp, or a non-functional promoter comprising a polynucleotide sequence of at least 10 bp. Those with skill in the art would appreciate that such a non-functional promoter would not be expected to drive expression of a coding sequence operably linked downstream of the minimal promoter. And, in the event that such a polynucleotide sequence did drive expression of a coding sequence operably linked downstream of the minimal promoter, that such a minimal promoter would function to drive expression of the coding sequence at low levels.

In other embodiments the non-functional promoter comprises a minimal promoter. Examples of a minimal promoter include truncated polynucleotide sequences of a full-length promoter. Such a minimal promoter drives lower levels of expression as compared to the full-length promoter. In certain aspects, the minimal promoter will include at least one promoter element. Non-limiting examples of such promoter elements include a TATA box, a CAAT box (also described as a CCAAT box), a transcription start site, an RNA polymerase binding site, or any combination thereof. The minimal promoter includes promoter elements necessary for RNA polymerase binding and initiation of transcription. For RNA polymerase II promoters the promoter is identified by a TATA-homologous sequences motif about 20 to 50 base pairs upstream of the transcription start site and a CAAT-homologous sequence motif about 50 to 120 base pairs upstream of the transcription start site. The TATA motif is the site where the TATA-binding-protein (TBP) as part of a complex of several polypeptides (TFIID complex) binds and productively interacts (directly or indirectly) with factors bound to other sequence elements of the promoter. This TFIID complex in turn recruits the RNA polymerase II complex to be positioned for the start of transcription generally 25 to 30 base pairs downstream of the TATA element and promotes elongation thus producing RNA molecules. The sequences around the start of transcription (designated INR) of some polIl genes seem to provide an alternate binding site for factors that also recruit members of the TFIID complex and thus “activate” transcription. These INR sequences are particularly relevant in promoters that lack functional TATA elements providing the core promoter binding sites for eventual transcription. It has been proposed that promoters containing both a functional TATA and INR motif are the most efficient in transcriptional activity. (Zenzie-Gregory et al, 1992. J. Biol. Chem. 267:2823-2830). Those with skill in the art appreciate that elements other than the TATA motif are required for accurate transcription. Such elements are often located upstream of the TATA motif and a subset may have homology to the consensus sequence CCAAT. An exemplary minimal promoter suitable for use in plants is the minimal promoter of the Ubiquitin-1 gene as disclosed in U.S. patent application Ser. No. 20/130,254943A1. An exemplary minimal promoter suitable for use in plants is the minimal promoter of the Arabidopsis thaliana Ubiquitin-10 gene or the minimal promoter Cassava Vein Mosaic Virus promoter as disclosed in U.S. patent application Ser. No. 20/160,130595A1. An exemplary minimal promoter suitable for use in plants is the truncated CaMV 35S promoter.

In further embodiments, known full-length plant promoters can be modified to be a non-functional promoter. Those with skill in the art would appreciate the molecular engineering of the polynucleotide sequence can result in the inactivation of a functional promoter to thereby render said promoter as a non-functional promoter. Any of a various number of manipulations can be utilized, non-limiting examples include truncations of a known plant promoter to produce a non-functional promoter, inversion of sequences of a known plant promoter to produce a non-functional promoter, or any other type of rearrangement of the polynucleotide sequence of a known plant-promoter to produce a non-functional promoter.

Known full-length plant promoters that can be modified to be a non-functional promoter include tissue specific promoters. Tissue specific promoters preferentially initiate transcription in certain tissues, such as stamen, anther, filament, and pollen, or developmental growth stages, such as sporogenous tissue, microspores, and microgametophyte. Such plant promoters are referred to as “tissue-preferred,” “cell-type-preferred,” or “growth-stage preferred.” Promoters which initiate transcription only in certain tissue are referred to as “tissue-specific.” Likewise, promoters which initiate transcription only at certain growth stages are referred to as “growth-stage-specific.” A “cell-type-specific” promoter drives expression only in certain cell types in one or more organs, for example, stamen cells, or individual cell types within the stamen such as anther, filament, or pollen cells.

Known full-length plant promoters that can be modified to be a non-functional promoter include male-fertility tissue-preferred or tissue-specific promoter promoters. One such promoter is the 5126 promoter, which preferentially directs expression of the polynucleotide to which it is linked to male tissue of the plants, as described in U.S. Pat. Nos. 5,837,851 and 5,689,051. Other examples include the maize Ms45 promoter described at U.S. Pat. No. 6,037,523; SF3 promoter described at U.S. Pat. No. 6,452,069; the BS92-7 promoter described at WO 02/063021; an SGB6 regulatory element described at U.S. Pat. No. 5,470,359; the TA29 promoter (Koltunow, et al., (1990) Plant Cell 2:1201-1224; Nature 347:737 (1990); Goldberg, et al., (1993) Plant Cell 5:1217-1229 and U.S. Pat. No. 6,399,856); an SB200 gene promoter (WO 2002/26789), a PG47 gene promoter (U.S. Pat. No. 5,412,085; U.S. Pat. No. 5,545,546; Plant J 3(2):261-271 (1993)), a G9 gene promoter (U.S. Pat. Nos. 5,837,850; 5,589,610); the type 2 metallothionein-like gene promoter (Charbonnel-Campaa, et al., Gene (2000) 254:199-208); the Brassica Bca9 promoter (Lee, et al., (2003) Plant Cell Rep. 22:268-273); the ZM13 promoter (Hamilton, et al., (1998) Plant Mol. Biol. 38:663-669); actin depolymerizing factor promoters (such as Zmabp1, Zmabp2; see, for example Lopez, et al., (1996) Proc. Natl. Acad. Sci. USA 93:7415-7420); the promoter of the maize pectin methylesterase-like gene, ZmC5 (Wakeley, et al., (1998) Plant Mol. Biol. 37:187-192); the profilin gene promoter Zmprol (Kovar, et al., (2000) The Plant Cell 12:583-598); the sulphated pentapeptide phytosulphokine gene ZmPSK1 (Lorbiecke, et al., (2005) Journal of Experimental Botany 56 (417): 1805-1819); the promoter of the calmodulin binding protein Mpcbp (Reddy, et al., (2000) J. Biol. Chem. 275 (45): 35457-70).

Known full-length plant promoters that can be modified to be a non-functional promoter include constitutive promoters. Constitutive promoters include, for example, the promoter of the Rsyn7 promoter and other constitutive promoters disclosed in WO 99/43838 and U.S. Pat. No. 6,072,050; the CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Known full-length plant promoters that can be modified to be a non-functional promoter include seed-preferred promoters. “Seed-preferred” promoters include both those promoters active during seed development, such as promoters of seed storage proteins, as well as those promoters active during seed germination. See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is an endosperm-specific promoter. Globulin-1 (Glob-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, globulin 1, etc. See also WO 00/12733, where seed-preferred promoters from end1 and end2 genes are disclosed. Additional embryo specific promoters are disclosed in Sato et al. (1996) Proc. Natl. Acad. Sci. 93:8117-8122; Nakase et al. (1997) Plant J 12:235-46; and Postma-Haarsma et al. (1999) Plant Mol. Biol. 39:257-71. Additional endosperm specific promoters are disclosed in Albani et al. (1984) EMBO 3:1405-15; Albani et al. (1999) Theor. Appl. Gen. 98:1253-62; Albani et al. (1993) Plant J. 4:343-55; Mena et al. (1998) The Plant Journal 116:53-62, and Wu et al. (1998) Plant Cell Physiology 39:885-889.

Known full-length plant promoters that can be modified to be a non-functional promoter include dividing cell or meristematic tissue-preferred promoters. Dividing cell or meristematic tissue-preferred promoters have been disclosed in Ito et al. (1994) Plant Mol. Biol. 24:863-878; Reyad et al. (1995) Mo. Gen. Genet. 248:703-711; Shaul et al. (1996) Proc. Natl. Acad. Sci. 93:4868-4872; Ito et al. (1997) Plant J. 11:983-992; and Trehin et al. (1997) Plant Mol. Biol. 35:667-672.

Known full-length plant promoters that can be modified to be a non-functional promoter include stress inducible promoters. Stress inducible promoters include salt/water stress-inducible promoters such as P5CS (Zang et al. (1997) Plant Sciences 129:81-89); cold-inducible promoters, such as, cor15a (Hajela et al. (1990) Plant Physiol. 93:1246-1252), cor15b (Wlihelm et al. (1993) Plant Mol Biol 23:1073-1077), wsc120 (Ouellet et al. (1998) FEBS Lett. 423-324-328), ci7 (Kirch et al. (1997) Plant Mol Biol. 33:897-909), ci21A (Schneider et al. (1997) Plant Physiol. 113:335-45); drought-inducible promoters, such as, Trg-31 (Chaudhary et al (1996) Plant Mol. Biol. 30:1247-57), rd29 (Kasuga et al. (1999) Nature Biotechnology 18:287-291); osmotic inducible promoters, such as, Rab17 (Vilardell et al. (1991) Plant Mol. Biol. 17:985-93) and osmotin (Raghothama et al. (1993) Plant Mol Biol 23:1117-28); and, heat inducible promoters, such as, heat shock proteins (Barros et al. (1992) Plant Mol. 19:665-75; Marrs et al. (1993) Dev. Genet. 14:27-41), and smHSP (Waters et al. (1996) J. Experimental Botany 47:325-338). Other stress-inducible promoters include rip2 (U.S. Pat. No. 5,332,808 and U.S. Publication No. 2003/0217393) and rd29A (Yamaguchi-Shinozaki et al. (1993) Mol. Gen. Genetics 236:331-340).

In other aspects the engineering of a site-specific promoter activation complex within proximity of a non-functional promoter results in higher levels of expression of the non-functional promoter. In certain embodiments the non-functional promoter drives low levels of expression of an operably linked coding sequence. In aspects thereof the low level of expression results in the production of a functional end-product (e.g., an mRNA, guide RNA, or a protein) that is not detectable through established molecular detection methods, or is detectable at low basal levels using such molecular detection methods. In other aspects the low level of expression results in the production of a functional end-product (e.g., an mRNA, guide RNA, or a protein) that does not produce any biological effect on the plant cell. As a non-limiting example, the low level expression of a coding sequence that encodes an antibiotic or herbicidal selectable marker would fail to protect the cell from injury or death when the cell is treated with an antibiotic or herbicide. In further aspects the low level of expression results in no production of any functional end-product (e.g., an mRNA, guide RNA, or a protein).

In further aspect the proximity of the site-specific promoter activation complex to the non-functional promoter results in higher levels of expression of the non-functional promoter. In an embodiment the site-specific promoter activation complex is designed to bind upstream polynucleotide sequence of the non-functional promoter. The site-specific promoter activation complex may be located within a proximity of at least 1 bp, at least 10 bp, at least 20 bp, at least 30 bp, at least 40 bp, at least 50 bp, at least 60 bp, at least 70 bp, at least 80 bp, at least 90 bp, at least 100 bp, at least 125 bp, at least 150 bp, at least 175 bp, at least 200 bp, at least 250 bp, at least 300 bp, at least 350 bp, at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, at least 1000 bp, at least 1100 bp, at least 1200 bp, at least 1300 bp, at least 1400 bp, at least 1500 bp, at least 2000 bp, at least 2500 bp, at least 3000 bp, at least 3500 bp, at least 4000 bp, at least 4500 bp, at least 5000 bp, at least 5500 bp, at least 6000 bp, at least 6500 bp, at least 7500 bp, at least 8000 bp, at least 8500 bp, at least 9000 bp, at least 9500 bp, or at least 10,000 bp to the non-functional promoter. Ideally, the spacing proximity is determined for the highest level of expression of the coding sequence.

In additional aspects the engineering of a site-specific promoter activation complex within proximity of a non-functional promoter results in higher levels of expression of the non-functional promoter. In turn the increased expression of the non-functional promoter drives expression of the coding sequence. In certain aspects the high level of expression results in the production of a functional end-product (e.g., an mRNA, guide RNA, or a protein) that is detectable through established molecular detection methods. In other aspects the high level of expression results in the production of a functional end-product (e.g., an mRNA, guide RNA, or a protein) that produces a measurable or observable biological effect on the plant cell. As a non-limiting example, the high level expression of a coding sequence that encodes an antibiotic or herbicidal selectable marker would protect the cell from injury or death when the cell is treated with an antibiotic or herbicide. In further aspects the site-specific promoter activation complex drives expression of the non-functional promoter at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97.5%, at least 99%, or 100% higher. In further aspects the site-specific promoter activation complex drives expression of the non-functional promoter at least 1, at least 1.5-fold, at least 2-fold, at least 3-fold, at least 4-fold, at least 5-fold, at least 10-fold, at least 15-fold, at least 20-fold, at least 25-fold, or at least 50-fold higher. In some aspects the site-specific promoter activation complex drives expression of the coding sequence by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 97.5%, at least 99%, or 100% higher. In additional aspects the site-specific promoter activation complex drives expression of the coding sequence by at least 1, at least 1.5-fold, at least 2-fold, at least 3-fold, at least 4-fold, at least 5-fold, at least 10-fold, at least 15-fold, at least 20-fold, at least 25-fold, or at least 50-fold higher.

In additional aspects the engineering of a site-specific promoter activation complex within proximity of a non-functional promoter results in higher levels of expression of the non-functional promoter. In turn the increased expression of the non-functional promoter drives expression of the coding sequence. In certain aspects the site-specific promoter activation complex is bound to the genomic DNA upstream of the non-functional promoter. In another aspect the site-specific promoter activation complex is bound to the genomic DNA downstream of the non-functional promoter. In some aspects the multiple copies of the site-specific promoter activation complex is bound to the genomic DNA upstream of the non-functional promoter. Accordingly, there may be 1, 2, 3, 4, 5, 6 7, 8, 9, 10 or more site-specific promoter activation complexes that are bound to the genomic DNA upstream of the non-functional promoter. In another aspect the site-specific promoter activation complex is bound to the genomic DNA downstream of the non-functional promoter using a covalent bond, a hydrogen bond, or a bond that utilizes Van der Waals forces.

### Site-Specific Promoter Activation Complex

In further aspects the site-specific promoter activation complex comprises at least one activation domain operably linked to a site-specific binding protein. The activation domain may be fused to the site-specific binding protein through various means know to those with skill in the art. For example, the polynucleotide coding sequence encoding a site-specific binding protein can be engineered to include the coding sequence of the activation domain. This chimeric molecule includes the coding sequence for the site-specific binding protein in-frame with the coding sequence. Upon translation of the coding sequence a chimeric protein is produced that comprises the site-specific binding protein operably linked to the activation domain, thereby producing a site-specific promoter activation complex.

For example, a VP16 activation domain can be fused to the C-terminus of a deactivated Cas9 molecule. Another example was the fusion of four tandem repeats of the VP16 activation domain to the Cas9 molecule to produce a deactivated Cas9-VP64 molecule. Other site-specific promoter activation complexes are known to those with skill in the art. As a non-limiting example fusions of a deactivated Cas9 site-specific binding protein to the P65 activation domain, the EDLL activation domain, TAL activation domain, and CBF1 activation domain are described in the art. Other site-specific binding proteins have been operably linked to an activation domain. In another aspect zinc fingers, TALENs, and meganucleases can be operably linked to the activation domain. In further aspects the activation domain is operably linked to the site-specific binding protein to produce a site-specific promoter activation complex, wherein the activation domain is selected from RTA, TFIIA, TFIIB, TFIID, TFIIE, TFIIF, TFIIH, VP64, VP16, VP160, GAL4, EDLL, ERF2, CBF1, ORCA2, DREB1A, LEAFY, or any combination thereof.

Multiple activation domains may be engineered to the site-specific binding protein when producing a site-specific promoter activation complex. In some embodiments, the site-specific promoter activation complex comprises at least one activation domain, at least two activation domains, at least three activation domains, at least four activation domains, at least five activation domains, at least six activation domains, at least seven activation domains, at least eight activation domains, at least nine activation domains, at least ten activation domains, or ten or more activation domains that are bound to the site-specific binding protein.

In some aspects the activation domain is operably linked to the site-specific binding protein as a continuous open reading frame. In an embodiment the activation domain is operably linked to the site-specific binding protein by directly fusing the activation domain to the site-specific binding protein. In an embodiment the activation domain is operably linked to the site-specific binding protein by directly engineering the activation domain within the coding sequence of the site-specific binding protein. In an embodiment the activation domain is operably linked to the site-specific binding protein by a polynucleotide linker with the activation domain to the site-specific binding protein. In other embodiments the activation domain is operably linked to the site-specific binding protein by an epitope/antibody interaction as for example with the Sun Tag system.

### Activation Domains

In other aspects the engineering of a site-specific promoter activation complex comprises at least one activation domain. Activation domains are proteins that function by recruiting through protein-protein interactions a number of different proteins involved in DNA transcription (e.g., nucleosome-remodeling complexes; the mediator complex; and general transcription factors, such as TFIIB, TBP, and TFIIH) to initiate or enhance the rate of transcription by affecting nucleosome assembly/disassembly, pre-initiation complex formation, promoter clearance, and/or the rate of elongation. The protein-protein interactions of transactivators and their binding partners involve discrete internal structural elements within the transactivators known as “transactivation domains (TADs).” TADs are thought to share little primary sequence homology and adopt a defined structure only upon binding to a target. Sigler (1988) Nature 333:210-2. Though acidic and hydrophobic residues within the TADs are thought to be important (see, e.g., Cress and Triezenberg (1991) Science 251 (4989): 87-90), the contribution of individual residues to activity is thought to be small. Hall and Struhl (2002) J Biol. Chem. 277:46043-50.

Various activation domains are known in the art. Typically, the activation domain recruits the transcription preinitiation complex to the promoter and for purposes of this disclosure to the non-functional promoter sequences. There are many types of activation domains known in the art. And, these activation domains may be obtained from viruses, plants, animals, or fungi. In certain aspects the activation domain is a transcription factor. Despite the source the activation domain functions to drive robust expression. In further aspects the activation domain is selected from RTA, TFIIA, TFIIB, TFIID, TFIIE, TFIIF, TFIIH, VP64, VP16, VP160, GAL4, EDLL, ERF2, CBF1, ORCA2, DREB1A, LEAFY, or any combination thereof. Other examples of activation domains for used in the disclosure include those provided in WO2021178162A1 (herein incorporated by reference in its entirety), WO2018183878A1 (herein incorporated by reference in its entirety), and WO2013116731A1 (herein incorporated by reference in its entirety). In further aspects there may be 1, 2, 3, 4, 5, 6 7, 8, 9, 10 or more activation domains engineered with a site-specific binding protein to comprise the site-specific promoter activation complexes.

### Site-Specific Binding Proteins

In other aspects the engineering of a site-specific promoter activation complex comprises a site-specific binding protein. In certain applications of this aspect the site-specific protein comprises either a CRISPR, a zinc finger protein, a TALEN protein, or a meganuclease protein. In further aspects the CRISPR, zinc finger protein, TALEN promoter, or meganuclease promoter are modified to bind DNA and to not cleave or break the phosphodiester bonds of the DNA. In some aspects the CRISPR, a zinc finger protein, a TALEN protein, or a meganuclease protein are mutagenized to bind DNA and to not cleave or break the phosphodiester bonds of the DNA. Such a site-specific binding protein comprises a catalytically inactive CRISPR, or a catalytically inactive TALEN.

### Zinc Fingers

As an example, the genetically modified cell or plant described herein, is generated using a zinc finger nuclease-mediated genome editing process. The process for editing a chromosomal sequence includes for example: (a) introducing into a cell at least one nucleic acid encoding a zinc finger nuclease that recognizes a target sequence in the chromosomal sequence and is able to cleave a site in the chromosomal sequence, and, optionally, (i) at least one donor polynucleotide that includes a sequence for integration flanked by an upstream sequence and a downstream sequence that exhibit substantial sequence identity with either side of the cleavage site, or (ii) at least one exchange polynucleotide comprising a sequence that is substantially identical to a portion of the chromosomal sequence at the cleavage site and which further comprises at least one nucleotide change; and (b) culturing the cell to allow expression of the zinc finger nuclease such that the zinc finger nuclease introduces a double-stranded break into the chromosomal sequence, and wherein the double-stranded break is repaired by (i) a non-homologous end-joining repair process such that an inactivating mutation is introduced into the chromosomal sequence, or (ii) a homology-directed repair process such that the sequence in the donor polynucleotide is integrated into the chromosomal sequence or the sequence in the exchange polynucleotide is exchanged with the portion of the chromosomal sequence. A zinc finger nuclease includes a DNA binding domain (i.e., zinc finger) and a cleavage domain (i.e., nuclease). The nucleic acid encoding a zinc finger nuclease may include DNA or RNA. Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Choo et al. (2000) Curr. Opin. Struct. Biol. 10:411-416; and Doyon et al. (2008) Nat. Biotechnol. 26:702-708; Santiago et al. (2008) Proc. Natl. Acad. Sci. USA 105:5809-5814; Urnov, et al., (2010) Nat Rev Genet. 11 (9): 636-46; and Shukla, et al., (2009) Nature 459 (7245): 437-41. An engineered zinc finger binding domain may have a novel binding specificity compared to a naturally-occurring zinc finger protein. As an example, the algorithm of described in U.S. Pat. No. 6,453,242 may be used to design a zinc finger binding domain to target a preselected sequence. Nondegenerate recognition code tables may also be used to design a zinc finger binding domain to target a specific sequence (Sera et al. (2002) Biochemistry 41:7074-7081). Tools for identifying potential target sites in DNA sequences and designing zinc finger binding domains may be used (Mandell et al. (2006) Nuc. Acid Res. 34: W516-W523; Sander et al. (2007) Nuc. Acid Res. 35: W599-W605).

An exemplary zinc finger DNA binding domain recognizes and binds a sequence having at least about 80% sequence identity with the desired target sequence. In other embodiments, the sequence identity may be about 81%, 82%, 83%, 84%, 85%, 86%, 87%, 88%, 89%, 90%, 91%, 92%, 93%, 94%, 95%, 96%, 97%, 98%, 99%, or 100%.

A zinc finger nuclease also includes a cleavage domain. The cleavage domain portion of the zinc finger nucleases may be obtained from any endonuclease or exonuclease. Non-limiting examples of endonucleases from which a cleavage domain may be derived include, but are not limited to, restriction endonucleases and homing endonucleases. See, for example, 2010-2011 Catalog, New England Biolabs, Beverly, Mass.; and Belfort et al. (1997) Nucleic Acids Res. 25:3379-3388. Additional enzymes that cleave DNA are known (e.g., S1 Nuclease; mung bean nuclease; pancreatic DNase I; micrococcal nuclease; yeast HO endonuclease). One or more of these enzymes (or functional fragments thereof) may be used as a source of cleavage domains.

### Meganucleases

Another example for genetically modifying the cell or plant described herein, is by using “custom” meganucleases produced to modify plant genomes (see e.g., WO 2009/114321; Gao et al. (2010) Plant Journal 1:176-187. The term “meganuclease” generally refers to a naturally-occurring homing endonuclease that binds double-stranded DNA at a recognition sequence that is greater than 12 base pairs and encompasses the corresponding intron insertion site. Naturally-occurring meganucleases can be monomeric (e.g., I-Scel) or dimeric (e.g., I-Crel). The term meganuclease, as used herein, can be used to refer to monomeric meganucleases, dimeric meganucleases, or to the monomers which associate to form a dimeric meganuclease.

Naturally-occurring meganucleases, for example, from the LAGLIDADG family, have been used to effectively promote site-specific genome modification in plants, yeast, Drosophila, mammalian cells and mice. Engineered meganucleases such as, for example, LIG-34 meganucleases, which recognize and cut a 22 basepair DNA sequence found in the genome of Zea mays (maize) are known (see e.g., US20110113509).

### TALENs

TAL (transcription activator-like) effectors from plant pathogenic Xanthomonas are important virulence factors that act as transcriptional activators in the plant cell nucleus, where they directly bind to DNA via a central domain of tandem repeats. A transcription activator-like (TAL) effector-DNA modifying enzymes (TALE or TALEN) are also used to engineer genetic changes. See e.g., US20110145940, Boch et al., (2009), Science 326(5959): 1509-12. Fusions of TAL effectors to the FokI nuclease provide TALENs that bind and cleave DNA at specific locations. Target specificity is determined by developing customized amino acid repeats in the TAL effectors.

### CRISPR System Components

Endonucleases are enzymes that cleave the phosphodiester bond within a polynucleotide chain, and include restriction endonucleases that cleave DNA at specific sites without damaging the bases. Examples of endonucleases include restriction endonucleases, meganucleases, TAL effector nucleases (TALENs), zinc finger nucleases, and Cas (CRISPR-associated) effector endonucleases.

Cas endonucleases, either as single effector proteins or in an effector complex with other components, unwind the DNA duplex at the target sequence and optionally cleave at least one DNA strand, as mediated by recognition of the target sequence by a polynucleotide (such as, but not limited to, a crRNA or guide RNA) that is in complex with the Cas effector protein. Such recognition and cutting of a target sequence by a Cas endonuclease typically occurs if the correct protospacer-adjacent motif (PAM) is located at or adjacent to the 3′ end of the DNA target sequence. Alternatively, a Cas endonuclease herein may lack DNA cleavage or nicking activity, but can still specifically bind to a DNA target sequence when complexed with a suitable RNA component. (See also U.S. Patent Application US20150082478 published 19 Mar. 2015 and US20150059010 published 26 Feb. 2015).

Cas endonucleases may occur as individual effectors (Class 2 CRISPR systems) or as part of larger effector complexes (Class I CRISPR systems).

Cas endonucleases that have been described include, but are not limited to, for example: Cas3 (a feature of Class 1 type I systems), Cas9 (a feature of Class 2 type II systems) and Cas12 (Cpf1) (a feature of Class 2 type V systems).

Cas endonucleases and effector proteins can be used for targeted genome editing (via simplex and multiplex double-strand breaks and nicks) and targeted genome regulation (via tethering of epigenetic effector domains to either the Cas protein or sgRNA. A Cas endonuclease can also be engineered to function as an RNA-guided recombinase, and via RNA tethers could serve as a scaffold for the assembly of multiprotein and nucleic acid complexes (Mali et al., 2013, Nature Methods Vol. 10:957-963).

Cas endonucleases, when complexed with a cognate guide RNA, recognize, bind to, and optionally nick or cleave a target polynucleotide.

A Cas endonuclease, effector protein, or functional fragment thereof, for use in the disclosed methods, can be isolated from a native source, or from, a recombinant source where the genetically modified host cell is modified to express the nucleic acid sequence encoding the protein. Alternatively, the Cas protein can be produced using cell free protein expression systems, or be synthetically produced. Effector Cas nucleases may be isolated and introduced into a heterologous cell, or may be modified from its native form to exhibit a different type or magnitude of activity than what it would exhibit in its native source. Such modifications include but are not limited to: fragments, variants, substitutions, deletions, and insertions.

Fragments and variants of Cas endonucleases and Cas effector proteins can be obtained via methods such as site-directed mutagenesis and synthetic construction. Methods for measuring endonuclease activity are well known in the art such as, but not limiting to, WO2013166113 published 7 Nov. 2013, WO2016186953 published 24 Nov. 2016, and WO2016186946 published 24 Nov. 2016.

The Cas endonuclease can comprise a modified form of the Cas polypeptide. The modified form of the Cas polypeptide can include an amino acid change (e.g., deletion, insertion, or substitution) that reduces the naturally-occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US20140068797 published 6 Mar. 2014). In some cases, the modified form of the Cas polypeptide has no substantial nuclease activity and is referred to as catalytically “inactivated Cas” or “deactivated Cas (dCas).” An inactivated Cas/deactivated Cas includes a deactivated Cas endonuclease (dCas). A catalytically inactive Cas effector protein can be fused to a heterologous sequence to induce or modify activity.

A Cas endonuclease can be part of a fusion protein comprising one or more heterologous protein domains (e.g., 1, 2, 3, or more domains in addition to the Cas protein). Such a fusion protein may comprise any additional protein sequence, and optionally a linker sequence between any two domains, such as between Cas and a first heterologous domain. Examples of protein domains that may be fused to a Cas protein herein include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas protein can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16.

A catalytically active and/or inactive Cas endonuclease can be fused to a heterologous sequence (US20140068797 published 6 Mar. 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA, such as a site-specific promoter activation complex. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A partially active or catalytically inactive Cas-alpha endonuclease can also be fused to another protein or domain, for example Clo51 or FokI nuclease, to generate double-strand breaks (Guilinger et al. Nature Biotechnology, volume 32, number 6, June 2014).

A catalytically active or inactive Cas protein, such as the Cas-alpha protein described herein, can also be in fusion with a molecule that directs editing of single or multiple bases in a polynucleotide sequence, for example a site-specific deaminase that can change the identity of a nucleotide, for example from C·G to T·A or an A·T to G·C (Gaudelli et al., Programmable base editing of A·T to G·C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016):420-4. A base editing fusion protein may comprise, for example, an active (double strand break creating), partially active (nickase) or deactivated (catalytically inactive) Cas-alpha endonuclease and a deaminase (such as, but not limited to, a cytidine deaminase, an adenine deaminase, APOBEC1, APOBEC3A, BE2, BE3, BE4, ABEs, or the like). Base edit repair inhibitors and glycosylase inhibitors (e.g., uracil glycosylase inhibitor (to prevent uracil removal)) are contemplated as other components of a base editing system, in some embodiments.

Cas endonucleases can be expressed and purified by methods known in the art, for example as described in WO/2016/186953 published 24 Nov. 2016.

### Guide Polynucleotides

The guide polynucleotide enables target recognition, binding, and optionally cleavage by the Cas endonuclease, and can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (US20150082478 published 19 Mar. 2015 and US20150059010 published 26 Feb. 2015). A guide polynucleotide may be engineered or synthetic.

The guide polynucleotide includes a chimeric non-naturally occurring guide RNA comprising regions that are not found together in nature (i.e., they are heterologous with each other). For example, a chimeric non-naturally occurring guide RNA comprising a first nucleotide sequence domain (referred to as Variable Targeting domain or VT domain) that can hybridize to a nucleotide sequence in a target DNA, linked to a second nucleotide sequence that can recognize the Cas endonuclease, such that the first and second nucleotide sequence are not found linked together in nature.

The guide polynucleotide can be a double molecule (also referred to as duplex guide polynucleotide) comprising a crNucleotide sequence (such as a crRNA) and a tracrNucleotide (such as a tracrRNA) sequence. In some cases, there is a linker polynucleotide that connects the crRNA and tracrRNA to form a single guide, for example an sgRNA.

The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA-combination sequences. In some embodiments, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in Bacteria and Archaea. The size of the fragment of the crRNA naturally occurring in Bacteria and Archaea that can be present in a crNucleotide disclosed herein can range from, but is not limited to, 2, 3, 4, 5, 6, 7, 8, 9,10, 11, 12, 13, 14, 15, 16, 17, 18, 19, 20 or more nucleotides. In some embodiments, a crRNA molecule is selected from the group consisting of: SEQID NOs: 57, 58, and 59.

In some embodiments the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one embodiment, the RNA that guides the RNA/Cas9 endonuclease complex is a duplexed RNA comprising a duplex crRNA-tracrRNA. The tracrRNA (trans-activating CRISPR RNA) comprises, in the 5′-to-3′ direction, (i) a sequence that anneals with the repeat region of CRISPR type II crRNA and (ii) a stem loop-comprising portion (Deltcheva et al., Nature 471:602-607). The duplex guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double-strand break) into the target site. (US20150082478 published 19 Mar. 2015 and US20150059010 published 26 Feb. 2015).

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide.

### Protospacer Adjacent Motif (PAM)

A “protospacer adjacent motif” (PAM) herein refers to a short nucleotide sequence adjacent to a target sequence (protospacer) that can be recognized (targeted) by a guide polynucleotide/Cas endonuclease system. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

A “randomized PAM” and “randomized protospacer adjacent motif” are used interchangeably herein, and refer to a random DNA sequence adjacent to a target sequence (protospacer) that is recognized (targeted) by a guide polynucleotide/Cas endonuclease system. The randomized PAM sequence can be of any length but is typically 1, 2, 3, 4, 5, 6, 7, 8, 9, 10, 11, 12, 13, 14, 15, 16, 17, 18, 19 or 20 nucleotides long. A randomized nucleotide includes anyone of the nucleotides A, C, G or T.

### Guide Polynucleotide/Cas Endonuclease Complexes

A guide polynucleotide/Cas endonuclease complex described herein is capable of recognizing, binding to, and optionally nicking, unwinding, or cleaving all or part of a target sequence.

A guide polynucleotide/Cas endonuclease complex that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild type Cas protein (e.g., a Cas protein disclosed herein), or a variant thereof retaining some or all activity in each endonuclease domain of the Cas protein, is a suitable example of a Cas endonuclease that can cleave both strands of a DNA target sequence.

A guide polynucleotide/Cas endonuclease complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. For example, a Cas9 nickase may comprise (i) a mutant, dysfunctional RuvC domain and (ii) a functional HNH domain (e.g., wild type HNH domain). As another example, a Cas9 nickase may comprise (i) a functional RuvC domain (e.g., wild type RuvC domain) and (ii) a mutant, dysfunctional HNH domain. Non-limiting examples of Cas9 nickases suitable for use herein are disclosed in U.S. Pat. No. 20,140,189896 published on 3 Jul. 2014. A pair of Cas nickases can be used to increase the specificity of DNA targeting. In general, this can be done by providing two Cas nickases that, by virtue of being associated with RNA components with different guide sequences, target and nick nearby DNA sequences on opposite strands in the region for desired targeting. Such nearby cleavage of each DNA strand creates a double-strand break (i.e., a DSB with single-stranded overhangs), which is then recognized as a substrate for non-homologous-end-joining, NHEJ (prone to imperfect repair leading to mutations) or homologous recombination, HR. Each nick in these embodiments can be at least about 5, between 5 and 10, at least 10, between 10 and 15, at least15, between 15 and 20, at least 20, between 20 and 30, at least 30, between 30 and 40, at least 40, between 40 and 50, at least 50, between 50 and 60, at least 60, between 60 and 70, at least 70, between 70 and 80, at least 80, between 80 and 90, at least 90, between 90 and 100, or 100 or greater (or any integer between 5 and 100) bases apart from each other, for example. One or two Cas nickase proteins herein can be used in a Cas nickase pair. For example, a Cas9 nickase with a mutant RuvC domain, but functioning HNH domain (i.e., Cas9 HNH+/RuvC−), can be used (e.g., Streptococcus pyogenes Cas9 HNH+/RuvC−). Each Cas9 nickase (e.g., Cas9 HNH+/RuvC−) can be directed to specific DNA sites nearby each other (up to 100 base pairs apart) by using suitable RNA components herein with guide RNA sequences targeting each nickase to each specific DNA site.

A guide polynucleotide/Cas endonuclease complex in certain embodiments can bind to a DNA target site sequence, but does not cleave any strand at the target site sequence. Such a complex may comprise a Cas protein in which all of its nuclease domains are mutant, dysfunctional. For example, a Cas9 protein that can bind to a DNA target site sequence, but does not cleave any strand at the target site sequence, may comprise both a mutant, dysfunctional RuvC domain and a mutant, dysfunctional HNH domain. A Cas protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein).

Modification of Genomes with Novel CRISPR-Cas System Components

As described herein, a guided Cas endonuclease can recognize, bind to a DNA target sequence and introduce a single strand (nick) or double-strand break. Once a single or double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements (such as chromosomal translocations) are possible (Siebert and Puchta, 2002, Plant Cell 14:1121-31; Pacher et al., 2007, Genetics 175:21-9).

DNA double-strand breaks appear to be an effective factor to stimulate homologous recombination pathways (Puchta et al., (1995) Plant Mol Biol 28:281-92; Tzfira and White, (2005) Trends Biotechnol 23:567-9; Puchta, (2005) J Exp Bot 56:1-14). Using DNA-breaking agents, a two- to nine-fold increase of homologous recombination was observed between artificially constructed homologous DNA repeats in plants (Puchta et al., (1995) Plant Mol Biol 28:281-92). In maize protoplasts, experiments with linear DNA molecules demonstrated enhanced homologous recombination between plasmids (Lyznik et al., (1991) Mol Gen Genet 230:209-18).

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. PNAS (0027-8424), 111 (10), p. E924-E932).

Alteration of the genome of a prokaryotic and eukaryotic cell or organism cell, for example, through homologous recombination (HR), is a powerful tool for genetic engineering. Homologous recombination has been demonstrated in plants (Halfter et al., (1992) Mol Gen Genet 231:186-93) and insects (Dray and Gloor, 1997, Genetics 147:689-99). Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86). In the filamentous fungus Aspergillus nidulans, gene replacement has been accomplished with as little as 50 bp flanking homology (Chaveroche et al., (2000) Nucleic Acids Res 28:e97). Targeted gene replacement has also been demonstrated in the ciliate Tetrahymena thermophila (Gaertig et al., (1994) Nucleic Acids Res 22:5391-8). In mammals, homologous recombination has been most successful in the mouse using pluripotent embryonic stem cell lines(ES) that can be grown in culture, transformed, selected and introduced into a mouse embryo (Watson et al., 1992, Recombinant DNA, 2nd Ed., Scientific American Books distributed by WH Freeman & Co.).

### Gene Targeting

The guide polynucleotide/Cas systems described herein can be used for gene targeting. In general, DNA targeting can be performed by cleaving one or both strands at a specific polynucleotide sequence in a cell with a Cas protein associated with a suitable polynucleotide component. Once a single or double-strand break is induced in the DNA, the cell's DNA repair mechanism is activated to repair the break via nonhomologous end-joining (NHEJ) or Homology-Directed Repair (HDR) processes which can lead to modifications at the target site.

The length of the DNA sequence at the target site can vary, and includes, for example, target sites that are at least 12, 13, 14, 15, 16, 17, 18, 19, 20, 21, 22, 23, 24, 25, 26, 27, 28, 29, 30, or more than 30 nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease.

Assays to measure the single or double-strand break of a target site by an endonuclease are known in the art and generally measure the overall activity and specificity of the agent on DNA substrates comprising recognition sites.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas endonuclease complex to a unique DNA target site.

### Gene Editing

The process for editing a genomic sequence combining DSB and modification templates generally comprises: introducing into a host cell a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB. Genome editing using DSB-inducing agents, such as Cas-gRNA complexes, has been described, for example in US20150082478 published on 19 Mar. 2015, WO2015026886 published on 26 Feb. 2015, WO2016007347 published 14 Jan. 2016, and WO/2016/025131 published on 18 Feb. 2016.

Some uses for guide RNA/Cas endonuclease systems have been described (see for example: US20150082478 A1 published 19 Mar. 2015, WO2015026886 published 26 Feb. 2015, and US20150059010 published 26 Feb. 2015) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

Described herein are methods for genome editing with a Cas endonuclease and complexes with a Cas endonuclease and a guide polynucleotide. Following characterization of the guide RNA and PAM sequence, components of the endonuclease and associated CRISPR RNA (crRNA) may be utilized to modify chromosomal DNA in other organisms including plants. To facilitate optimal expression and nuclear localization (for eukaryotic cells), the genes comprising the complex may be optimized as described in WO2016186953 published 24 Nov. 2016, and then delivered into cells as DNA expression cassettes by methods known in the art. The components necessary to comprise an active complex may also be delivered as RNA with or without modifications that protect the RNA from degradation or as mRNA capped or uncapped (Zhang, Y. et al., 2016, Nat. Commun. 7:12617) or Cas protein guide polynucleotide complexes (WO2017070032 published 27 Apr. 2017), or any combination thereof. Additionally, a part or part(s) of the complex and crRNA may be expressed from a DNA construct while other components are delivered as RNA with or without modifications that protect the RNA from degradation or as mRNA capped or uncapped (Zhang et al. 2016 Nat. Commun. 7:12617) or Cas protein guide polynucleotide complexes (WO2017070032 published 27 Apr. 2017) or any combination thereof. To produce crRNAs in-vivo, tRNA derived elements may also be used to recruit endogenous RNAses to cleave crRNA transcripts into mature forms capable of guiding the complex to its DNA target site, as described, for example, in WO2017105991 published 22 Jun. 2017. Nickase complexes may be utilized separately or concertedly to generate a single or multiple DNA nicks on one or both DNA strands. Furthermore, the cleavage activity of the Cas endonuclease may be deactivated by altering key catalytic residues in its cleavage domain (Sinkunas, T. et al., 2013, EMBO J. 32:385-394) resulting in a RNA guided helicase that may be used to enhance homology directed repair, induce transcriptional activation, or remodel local DNA structures. Moreover, the activity of the Cas cleavage and helicase domains may both be knocked-out and used in combination with other DNA cutting, DNA nicking, DNA binding, transcriptional activation, transcriptional repression, DNA remodeling, DNA deamination, DNA unwinding, DNA recombination enhancing, DNA integration, DNA inversion, and DNA repair agents.

The transcriptional direction of the tracrRNA for the CRISPR-Cas system (if present) and other components of the CRISPR-Cas system (such as variable targeting domain, crRNA repeat, loop, anti-repeat) can be deduced as described in WO2016186946 published 24 Nov. 2016, and WO2016186953 published 24 Nov. 2016.

As described herein, once the appropriate guide RNA requirement is established, the PAM preferences for each new system disclosed herein may be examined. If the cleavage complex results in degradation of the randomized PAM library, the complex can be converted into a nickase by disabling the ATPase dependent helicase activity either through mutagenesis of critical residues or by assembling the reaction in the absence of ATP as described previously (Sinkunas, T. et al., 2013, EMBO J. 32:385-394). Two regions of PAM randomization separated by two protospacer targets may be utilized to generate a double-stranded DNA break which may be captured and sequenced to examine the PAM sequences that support cleavage by the respective complex.

In one embodiment, the invention describes a method for modifying a target site in the genome of a cell, the method comprising introducing into a cell at least one PGEN described herein, and identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, the chemical alteration of at least one nucleotide, and (v) any combination of (i)-(iv).

The nucleotide to be edited can be located within or outside a target site recognized and cleaved by a Cas endonuclease. In one embodiment, the at least one nucleotide modification is not a modification at a target site recognized and cleaved by a Cas endonuclease. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

A knock-out may be produced by an indel (insertion or deletion of nucleotide bases in a target DNA sequence through NHEJ), or by specific removal of sequence that reduces or completely destroys the function of sequence at or near the targeting site.

A guide polynucleotide/Cas endonuclease induced targeted mutation can occur in a nucleotide sequence that is located within or outside a genomic target site that is recognized and cleaved by the Cas endonuclease.

The method for editing a nucleotide sequence in the genome of a cell can be a method without the use of an exogenous selectable marker by restoring function to a non-functional gene product.

In one embodiment, the invention describes a method for modifying a target site in the genome of a cell, the method comprising introducing into a cell at least one PGEN described herein and at least one donor DNA, wherein said donor DNA comprises a polynucleotide of interest, and optionally, further comprising identifying at least one cell that said polynucleotide of interest integrated in or near said target site.

In one aspect, the methods disclosed herein may employ homologous recombination (HR) to provide integration of the polynucleotide of interest at the target site.

Various methods and compositions can be employed to produce a cell or organism having a polynucleotide of interest inserted in a target site via activity of a CRISPR-Cas system component described herein. In one method described herein, a polynucleotide of interest is introduced into the organism cell via a donor DNA construct. As used herein, “donor DNA” is a DNA construct that comprises a polynucleotide of interest to be inserted into the target site of a Cas endonuclease. The donor DNA construct further comprises a first and a second region of homology that flank the polynucleotide of interest. The first and second regions of homology of the donor DNA share homology to a first and a second genomic region, respectively, present in or flanking the target site of the cell or organism genome.

The donor DNA can be tethered to the guide polynucleotide. Tethered donor DNAs can allow for co-localizing target and donor DNA, useful in genome editing, gene insertion, and targeted genome regulation, and can also be useful in targeting post-mitotic cells where function of endogenous HR machinery is expected to be highly diminished (Mali et al., 2013, Nature Methods Vol. 10:957-963).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

Episomal DNA molecules can also be ligated into the double-strand break, for example, integration of T-DNAs into chromosomal double-strand breaks (Chilton and Que, (2003) Plant Physiol 133:956-65; Salomon and Puchta, (1998) EMBO J. 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

In one embodiment, the disclosure comprises a method for editing a nucleotide sequence in the genome of a cell, the method comprising introducing into at least one PGEN described herein, and a polynucleotide modification template, wherein said polynucleotide modification template comprises at least one nucleotide modification of said nucleotide sequence, and optionally further comprising selecting at least one cell that comprises the edited nucleotide sequence.

The guide polynucleotide/Cas endonuclease system can be used in combination with at least one polynucleotide modification template to allow for editing (modification) of a genomic nucleotide sequence of interest. (See also US20150082478, published 19 Mar. 2015 and WO2015026886 published 26 Feb. 2015).

Polynucleotides of interest and/or traits can be stacked together in a complex trait locus as described in WO2012129373 published 27 Sep. 2012, and in WO2013112686, published 1 Aug. 2013. The guide polynucleotide/Cas9 endonuclease system described herein provides for an efficient system to generate double-strand breaks and allows for traits to be stacked in a complex trait locus.

A guide polynucleotide/Cas system as described herein, mediating gene targeting, can be used in methods for directing heterologous gene insertion and/or for producing complex trait loci comprising multiple heterologous genes in a fashion similar as disclosed in WO2012129373 published 27 Sep. 2012, where instead of using a double-strand break inducing agent to introduce a gene of interest, a guide polynucleotide/Cas system as disclosed herein is used. By inserting independent transgenes within 0.1, 0.2, 0.3, 0.4, 0.5, 1.0, 2, or even 5 centimorgans (cM) from each other, the transgenes can be bred as a single genetic locus (see, for example, US20130263324 published 3 Oct. 2013 or WO2012129373 published 14 Mar. 2013). After selecting a plant comprising a transgene, plants comprising (at least) one transgenes can be crossed to form an F1 that comprises both transgenes. In progeny from these F1 (F2 or BC1) 1/500 progeny would have the two different transgenes recombined onto the same chromosome. The complex locus can then be bred as single genetic locus with both transgene traits. This process can be repeated to stack as many traits as desired.

Further uses for guide RNA/Cas endonuclease systems have been described (See for example: US20150082478 published 19 Mar. 2015, WO2015026886 published 26 Feb. 2015, US20150059010 published 26 Feb. 2015, WO2016007347 published 14 Jan. 2016, and PCT application WO2016025131 published 18 Feb. 2016) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Resulting characteristics from the gene editing compositions and methods described herein may be evaluated. Chromosomal intervals that correlate with a phenotype or trait of interest can be identified. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for a particular trait. In one embodiment, the chromosomal interval comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifies the same QTL or two different QTL. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

Introduction of CRISPR-Cas System Components into a Cell

The methods and compositions described herein do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex (PGEN, RGEN) to the cell.

Methods for introducing polynucleotides or polypeptides or a polynucleotide-protein complex into cells or organisms are known in the art including, but not limited to, microinjection, electroporation, stable transformation methods, transient transformation methods, ballistic particle acceleration (particle bombardment), whiskers mediated transformation, Agrobacterium-mediated transformation, direct gene transfer, viral-mediated introduction, transfection, transduction, cell-penetrating peptides, mesoporous silica nanoparticle (MSN)-mediated direct protein delivery, topical applications, sexual crossing, sexual breeding, and any combination thereof.

For example, the guide polynucleotide (guide RNA, crNucleotide+tracrNucleotide, guide DNA and/or guide RNA-DNA molecule) can be introduced into a cell directly (transiently) as a single stranded or double stranded polynucleotide molecule. The guide RNA (or crRNA+tracrRNA) can also be introduced into a cell indirectly by introducing a recombinant DNA molecule comprising a heterologous nucleic acid fragment encoding the guide RNA (or crRNA+tracrRNA), operably linked to a specific promoter that is capable of transcribing the guide RNA (crRNA+tracrRNA molecules) in said cell. The specific promoter can be, but is not limited to, a RNA polymerase III promoter, which allow for transcription of RNA with precisely defined, unmodified, 5′- and 3′-ends (Ma et al., 2014, Mol. Ther. Nucleic Acids 3: e161; DiCarlo et al., 2013, Nucleic Acids Res. 41:4336-4343; WO2015026887, published 26 Feb. 2015). Any promoter capable of transcribing the guide RNA in a cell can be used and includes a heat shock/heat inducible promoter operably linked to a nucleotide sequence encoding the guide RNA.

Plant cells differ from animal cells (such as human cells), fungal cells (such as yeast cells) and protoplasts, including for example plant cells comprise a plant cell wall which may act as a barrier to the delivery of components.

Delivery of the Cas endonuclease, and/or the guide RNA, and/or a ribonucleoprotein complex, and/or a polynucleotide encoding any one or more of the preceding, into plant cells can be achieved through methods known in the art, for example but not limited to: Rhizobiales-mediated transformation (e.g., Agrobacterium, Ochrobactrum), particle mediated delivery (particle bombardment), polyethylene glycol (PEG)-mediated transfection (for example to protoplasts), electroporation, cell-penetrating peptides, or mesoporous silica nanoparticle (MSN)-mediated direct protein delivery.

The Cas endonuclease, such as the Cas endonuclease described herein, can be introduced into a cell by directly introducing the Cas polypeptide itself (referred to as direct delivery of Cas endonuclease), the mRNA encoding the Cas protein, and/or the guide polynucleotide/Cas endonuclease complex itself, using any method known in the art. The Cas endonuclease can also be introduced into a cell indirectly by introducing a recombinant DNA molecule that encodes the Cas endonuclease. The endonuclease can be introduced into a cell transiently or can be incorporated into the genome of the host cell using any method known in the art. Uptake of the endonuclease and/or the guided polynucleotide into the cell can be facilitated with a Cell Penetrating Peptide (CPP) as described in WO2016073433 published 12 May 2016. Any promoter capable of expressing the Cas endonuclease in a cell can be used and includes a heat shock/heat inducible promoter operably linked to a nucleotide sequence encoding the Cas endonuclease.

Direct delivery of a polynucleotide modification template into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein delivery can be successfully used for delivering a polynucleotide modification template in eukaryotic cells, such as plant cells.

The donor DNA can be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome.

Direct delivery of any one of the guided Cas system components can be accompanied by direct delivery (co-delivery) of other mRNAs that can promote the enrichment and/or visualization of cells receiving the guide polynucleotide/Cas endonuclease complex components. For example, direct co-delivery of the guide polynucleotide/Cas endonuclease components (and/or guide polynucleotide/Cas endonuclease complex itself) together with mRNA encoding phenotypic markers (such as but not limiting to transcriptional activators such as CRC (Bruce et al. 2000 The Plant Cell 12:65-79) can enable the selection and enrichment of cells without the use of an exogenous selectable marker by restoring function to a non-functional gene product as described in WO2017070032 published 27 Apr. 2017.

Introducing a guide RNA/Cas endonuclease complex described herein, (representing the cleavage ready complex described herein) into a cell includes introducing the individual components of said complex either separately or combined into the cell, and either directly (direct delivery as RNA for the guide and protein for the Cas endonuclease and protein subunits, or functional fragments thereof) or via recombination constructs expressing the components (guide RNA, Cas endonuclease, protein subunits, or functional fragments thereof). Introducing a guide RNA/Cas endonuclease complex (RGEN) into a cell includes introducing the guide RNA/Cas endonuclease complex as a ribonucleotide-protein into the cell. The ribonucleotide-protein can be assembled prior to being introduced into the cell as described herein. The components comprising the guide RNA/Cas endonuclease ribonucleotide protein (at least one Cas endonuclease, at least one guide RNA, at least one protein subunit) can be assembled in vitro or assembled by any means known in the art prior to being introduced into a cell (targeted for genome modification as described herein).

Direct delivery of the RGEN ribonucleoprotein, allows for genome editing at a target site in the genome of a cell which can be followed by rapid degradation of the complex, and only a transient presence of the complex in the cell. This transient presence of the RGEN complex may lead to reduced off-target effects. In contrast, delivery of RGEN components (guide RNA, Cas9 endonuclease) via plasmid DNA sequences can result in constant expression of RGENs from these plasmids which can intensify off target effects (Cradick, T. J. et al. (2013) Nucleic Acids Res 41:9584-9592; Fu, Y et al. (2014) Nat. Biotechnol. 31:822-826).

Direct delivery can be achieved by combining any one component of the guide RNA/Cas endonuclease complex (RGEN), representing the cleavage ready complex described herein, (such as at least one guide RNA, at least one Cas protein, and optionally one additional protein), with a delivery matrix comprising a microparticle (such as but not limited to of a gold particle, tungsten particle, and silicon carbide whisker particle) (see also WO2017070032 published 27 Apr. 2017). The delivery matrix may comprise any one of the components, such as the Cas endonuclease, that is attached to a solid matrix (e.g., a particle for bombardment).

In one aspect the guide polynucleotide/Cas endonuclease complex, is a complex wherein the site-specific promoter activation complex protein forming the site-specific promoter activation complex are introduced into the cell as RNA and protein, respectively.

In one aspect the site-specific promoter activation complex, is a complex wherein the activation domain and the site-specific binding protein and the at least one protein subunit of a complex forming the site-specific promoter activation complex are introduced into the cell as RNA and proteins, respectively.

In one aspect the site-specific promoter activation complex, is a complex wherein the activation domain and the site-specific binding protein and the at least one protein subunit of a complex forming the site-specific promoter activation complex are preassembled in vitro and introduced into the cell as a ribonucleotide-protein complex.

Protocols for introducing polynucleotides, polypeptides or polynucleotide-protein complexes (PGEN, RGEN) into eukaryotic cells, such as plants or plant cells are known and include microinjection (Crossway et al., (1986) Biotechniques 4:320-34 and U.S. Pat. No. 6,300,543), meristem transformation (U.S. Pat. No. 5,736,369), electroporation (Riggs et al., (1986) Proc. Natl. Acad. Sci. USA 83:5602-6, Agrobacterium-mediated transformation (U.S. Pat. Nos. 5,563,055 and 5,981,840), whiskers mediated transformation (Ainley et al. 2013, Plant Biotechnology Journal 11:1126-1134; Shaheen A. and M. Arshad 2011 Properties and Applications of Silicon Carbide (2011), 345-358 Editor(s): Gerhardt, Rosario. Publisher:InTech, Rijeka, Croatia. CODEN:69PQBP; ISBN:978-953-307-201-2), direct gene transfer (Paszkowski et al., (1984) EMBO J 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin); McCabe et al., (1988) Biotechnology 6:923-6; Weissinger et al., (1988) Ann Rev Genet 22:421-77; Sanford et al., (1987) Particulate Science and Technology 5:27-37 (onion); Christou et al., (1988) Plant Physiol 87:671-4 (soybean); Finer and McMullen, (1991) In vitro Cell Dev Biol 27P: 175-82 (soybean); Singh et al., (1998) Theor Appl Genet 96:319-24 (soybean); Datta et al., (1990) Biotechnology 8:736-40 (rice); Klein et al., (1988) Proc. Natl. Acad. Sci. USA 85:4305-9 (maize); Klein et al., (1988) Biotechnology 6:559-63 (maize); U.S. Pat. Nos. 5,240,855; 5,322,783 and 5,324,646; Klein et al., (1988) Plant Physiol 91:440-4 (maize); Fromm et al., (1990) Biotechnology 8:833-9 (maize); Hooykaas-Van Slogteren et al., (1984) Nature 311:763-4; U.S. Pat. No. 5,736,369 (cereals); Bytebier et al., (1987) Proc. Natl. Acad. Sci. USA 84:5345-9 (Liliaceae); De Wet et al., (1985) in The Experimental Manipulation of Ovule Tissues, ed. Chapman et al., (Longman, New York), pp. 197-209 (pollen); Kaeppler et al., (1990) Plant Cell Rep 9:415-8) and Kaeppler et al., (1992) Theor Appl Genet 84:560-6 (whisker-mediated transformation); D'Halluin et al., (1992) Plant Cell 4:1495-505 (electroporation); Li et al., (1993) Plant Cell Rep 12:250-5; Christou and Ford (1995) Annals Botany 75:407-13 (rice) and Osjoda et al., (1996) Nat Biotechnol 14:745-50 (maize via Agrobacterium tumefaciens).

Alternatively, polynucleotides may be introduced into plant or plant cells by contacting cells or organisms with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931.

The polynucleotide or recombinant DNA construct can be provided to or introduced into a prokaryotic and eukaryotic cell or organism using a variety of transient transformation methods. Such transient transformation methods include, but are not limited to, the introduction of the polynucleotide construct directly into the plant.

Nucleic acids and proteins can be provided to a cell by any method including methods using molecules to facilitate the uptake of anyone or all components of a guided Cas system (protein and/or nucleic acids), such as cell-penetrating peptides and nanocarriers. See also US20110035836 published 10 Feb. 2011, and EP2821486A1 published 7 Jan. 2015.

Other methods of introducing polynucleotides into a prokaryotic and eukaryotic cell or organism or plant part can be used, including plastid transformation methods, and the methods for introducing polynucleotides into tissues from seedlings or mature seeds.

Stable transformation is intended to mean that the nucleotide construct introduced into an organism integrates into a genome of the organism and is capable of being inherited by the progeny thereof. Transient transformation is intended to mean that a polynucleotide is introduced into the organism and does not integrate into a genome of the organism or a polypeptide is introduced into an organism. Transient transformation indicates that the introduced composition is only temporarily expressed or present in the organism.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

### Cells and Plants

The presently disclosed polynucleotides and polypeptides can be introduced into a cell. Cells include, but are not limited to, human, non-human, animal, mammalian, bacterial, fungal, insect, yeast, non-conventional yeast, and plant cells as well as plants and seeds produced by the methods described herein. Any plant can be used with the compositions and methods described herein, including monocot and dicot plants, and plant elements.

Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum species, for example Triticum aestivum, Triticum monococcum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses.

Examples of dicot plants that can be used include, but are not limited to, soybean (Glycine max), Brassica species (for example but not limited to: oilseed rape or Canola) (Brassica napus, B. campestris, Brassica rapa, Brassica. juncea), alfalfa (Medicago sativa),), tobacco (Nicotiana tabacum), Arabidopsis (Arabidopsis thaliana), sunflower (Helianthus annuus), cotton (Gossypium arboreum, Gossypium barbadense), and peanut (Arachis hypogaea), tomato (Solanum lycopersicum), potato (Solanum tuberosum.

Additional plants that can be used include safflower (Carthamus tinctorius), sweet potato (Ipomoea batatus), cassava (Manihot esculenta), coffee (Coffea spp.), coconut (Cocos nucifera), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), vegetables, ornamentals, and conifers.

Vegetables that can be used include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be used include pines such as loblolly pine (Pinus taeda), slash pine (Pinus elliotii), ponderosa pine (Pinus ponderosa), lodgepole pine (Pinus contorta), and Monterey pine (Pinus radiata); Douglas fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow cedar (Chamaecyparis nootkatensis).

In certain embodiments of the disclosure, a fertile plant is a plant that produces viable male and female gametes and is self-fertile. Such a self-fertile plant can produce a progeny plant without the contribution from any other plant of a gamete and the genetic material comprised therein. Other embodiments of the disclosure can involve the use of a plant that is not self-fertile because the plant does not produce male gametes, or female gametes, or both, that are viable or otherwise capable of fertilization.

The present disclosure finds use in the breeding of plants comprising one or more introduced traits, or edited genomes.

### Traits

In some aspects, the subject disclosure relates to identification and detection of coding sequence comprises an agronomic trait. In further aspects the economically important trait is selected from the group consisting of herbicide tolerance, disease resistance, insect or pest resistance, altered fatty acid, protein or carbohydrate metabolism, increased grain yield, increased oil, enhanced nutritional content, increased growth rates, enhanced stress tolerance, preferred maturity, enhanced organoleptic properties, altered morphological characteristics, and sterility.

### Insect Resistance

Various insect resistance genes can comprise a coding sequence. Exemplary insect resistance coding sequences are known in the art. As embodiments of insect resistance coding sequences that can be operably linked to the regulatory elements of the subject disclosure, the following traits are provided. Coding sequences that provide exemplary Lepidopteran insect resistance include: cry1A; cry1A.105; cry1Ab; cry1Ab (truncated); cry1Ab-Ac (fusion protein); cry1Ac (marketed as Widestrike®); cry1C; cry1F (marketed as Widestrike®); cry1Fa2; cry2Ab2; cry2Ae; cry9C; mocry1F; pinII (protease inhibitor protein); vip3A(a); and vip3Aa20. Coding sequences that provide exemplary Coleopteran insect resistance include: cry34Ab1 (marketed as Herculex®); cry35Ab1 (marketed as Herculex®); cry3A; cry3Bb1; dvsnf7; and mcry3A. Coding sequences that provide exemplary multi-insect resistance include ecry31.Ab. The above list of insect resistance genes is not meant to be limiting. Any insect resistance genes are encompassed by the present disclosure.

### Herbicide Tolerance

Various herbicide tolerance genes can comprise a coding sequence. Exemplary herbicide tolerance coding sequences are known in the art. As embodiments of herbicide tolerance coding sequences that can be operably linked to the regulatory elements of the subject disclosure, the following traits are provided. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to the gene regulatory elements of the subject disclosure. In an embodiment, selectable marker genes include, but are not limited to genes encoding glyphosate resistance genes include: mutant EPSPS genes such as 2mEPSPS genes, cp4 EPSPS genes, mEPSPS genes, dgt-28 genes; aroA genes; and glyphosate degradation genes such as glyphosate acetyl transferase genes (gat) and glyphosate oxidase genes (gox). These traits are currently marketed as Gly-Tol™, Optimum® GAT®, Agrisure® GT and Roundup Ready®. Resistance genes for glufosinate and/or bialaphos compounds include dsm-2, bar and pat genes. The bar and pat traits are currently marketed as LibertyLink®. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). These traits are marketed as Enlist® crop protection technology. Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csr1-2 genes, Sr-HrA genes, and surB genes. Some of the traits are marketed under the tradename Clearfield®. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83. ), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim M K, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560. ) Resistance genes for pyridinoxy or phenoxy proprionic acids and cyclohexones include the ACCase inhibitor-encoding genes (e.g., Acc1-S1, Acc1-S2 and Acc1-S3). Exemplary genes conferring resistance to cyclohexanediones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), Is · genes (tolerance to triazine), and nitrilase genes (tolerance to benzonitrile). The above list of herbicide tolerance genes is not meant to be limiting. Any herbicide tolerance genes are encompassed by the present disclosure.

### Agronomic Traits

Various agronomic trait genes can comprise a coding sequence. Exemplary agronomic trait coding sequences are known in the art. As embodiments of agronomic trait coding sequences that can be operably linked to the regulatory elements of the subject disclosure, the following traits are provided. Delayed fruit softening as provided by the pg genes inhibit the production of polygalacturonase enzyme responsible for the breakdown of pectin molecules in the cell wall, and thus causes delayed softening of the fruit. Further, delayed fruit ripening/senescence of acc genes act to suppress the normal expression of the native acc synthase gene, resulting in reduced ethylene production and delayed fruit ripening. Whereas, the accd genes metabolize the precursor of the fruit ripening hormone ethylene, resulting in delayed fruit ripening. Alternatively, the sam-k genes cause delayed ripening by reducing S-adenosylmethionine (SAM), a substrate for ethylene production. Drought stress tolerance phenotypes as provided by cspB genes maintain normal cellular functions under water stress conditions by preserving RNA stability and translation. Another example includes the FcBetA genes that catalyze the production of the osmoprotectant compound glycine betaine conferring tolerance to water stress. In addition, the RmBetA genes catalyze the production of the osmoprotectant compound glycine betaine conferring tolerance to water stress. Photosynthesis and yield enhancement is provided with the bbx32 gene that expresses a protein that interacts with one or more endogenous transcription factors to regulate the plant's day/night physiological processes. Ethanol production can be increase by expression of the amy797E genes that encode a thermostable alpha-amylase enzyme that enhances bioethanol production by increasing the thermostability of amylase used in degrading starch. Finally, modified amino acid compositions can result by the expression of the cordapA genes that encode a dihydrodipicolinate synthase enzyme that increases the production of amino acid lysine. The above list of agronomic trait coding sequences is not meant to be limiting. Any agronomic trait coding sequence is encompassed by the present disclosure.

### DNA Binding Proteins

Various DNA binding transgene/heterologous coding sequence genes/heterologous coding sequences can comprise a coding sequence. Exemplary DNA binding protein coding sequences are known in the art. As embodiments of DNA binding protein coding sequences that can be operably linked to the regulatory elements of the subject disclosure, the following types of DNA binding proteins can include; Zinc Fingers, TALENs, CRISPRS, and meganucleases. The above list of DNA binding protein coding sequences is not meant to be limiting. Any DNA binding protein coding sequences is encompassed by the present disclosure.

### Small RNA

Various small RNA sequences can comprise a coding sequence. Exemplary small RNA traits are known in the art. As embodiments of small RNA coding sequences that can be operably linked to the regulatory elements of the subject disclosure, the following traits are provided. For example, delayed fruit ripening/senescence of the anti-efe small RNA delays ripening by suppressing the production of ethylene via silencing of the ACO gene that encodes an ethylene-forming enzyme. The altered lignin production of ccomt small RNA reduces content of guanacyl (G) lignin by inhibition of the endogenous S-adenosyl-L-methionine: trans-caffeoyl CoA 3-O-methyltransferase (CCOMT gene). Further, the Black Spot Bruise Tolerance in Solanum verrucosum can be reduced by the Ppo5 small RNA which triggers the degradation of Ppo5 transcripts to block black spot bruise development. Also included is the dvsnf7 small RNA that inhibits Western Corn Rootworm with dsRNA containing a 240 bp fragment of the Western Corn Rootworm Snf7 gene. Modified starch/carbohydrates can result from small RNA such as the pPhL small RNA (degrades PhL transcripts to limit the formation of reducing sugars through starch degradation) and pR1 small RNA (degrades R1 transcripts to limit the formation of reducing sugars through starch degradation). Additional, benefits such as reduced acrylamide resulting from the asnl small RNA that triggers degradation of Asnl to impair asparagine formation and reduce polyacrylamide. Finally, the non-browning phenotype of pgas ppo suppression small RNA results in suppressing PPO to produce apples with a non-browning phenotype. The above list of small RNAs is not meant to be limiting. Any small RNA encoding sequences are encompassed by the present disclosure.

### Selectable Markers

Various selectable markers also described as reporter genes can comprise a coding sequence. Many methods are available to confirm expression of selectable markers in transformed plants, including for example DNA sequencing and PCR (polymerase chain reaction), Southern blotting, RNA blotting, immunological methods for detection of a protein expressed from the vector. But, usually the reporter genes are observed through visual observation of proteins that when expressed produce a colored product. Exemplary reporter genes are known in the art and encode β-glucuronidase (GUS), luciferase, green fluorescent protein (GFP), yellow fluorescent protein (YFP, Phi-YFP), red fluorescent protein (DsRFP, RFP, etc), β-galactosidase, and the like (See Sambrook, et al., Molecular Cloning: A Laboratory Manual, Third Edition, Cold Spring Harbor Press, N.Y., 2001, the content of which is incorporated herein by reference in its entirety).

Selectable marker genes are utilized for selection of transformed cells or tissues. Selectable marker genes include genes encoding antibiotic resistance, such as those encoding neomycin phosphotransferase II (NEO), spectinomycin/streptinomycin resistance (AAD), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

In an embodiment, herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridichromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Acc1-S1, Acc1-S2 and Acc1-S3. In an embodiment, herbicides can inhibit photosynthesis, including triazine (psbA and 1s+ genes) or benzonitrile (nitrilase gene). Futhermore, such selectable markers can include positive selection markers such as phosphomannose isomerase (PMI) enzyme.

In an embodiment, selectable marker genes include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). An embodiment also includes selectable marker genes encoding resistance to: chloramphenicol; methotrexate; hygromycin; spectinomycin; bromoxynil; glyphosate; and phosphinothricin. The above list of selectable marker genes is not meant to be limiting. Any reporter or selectable marker gene are encompassed by the present disclosure.

In some embodiments the coding sequences are synthesized for optimal expression in a plant. For example, in an embodiment, a coding sequence of a gene has been modified by codon optimization to enhance expression in plants. An insecticidal resistance transgene, an herbicide tolerance transgene, a nitrogen use efficiency transgene, a water use efficiency transgene, a nutritional quality transgene, a DNA binding transgene, or a selectable marker transgene/heterologous coding sequence can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest. In an embodiment, a coding sequence, gene, heterologous coding sequence or transgene/heterologous coding sequence is designed to be expressed in plants at a higher level resulting in higher transformation efficiency. Methods for plant optimization of genes are well known. Guidance regarding the optimization and production of synthetic DNA sequences can be found in, for example, WO2013016546, WO2011146524, WO1997013402, U.S. Pat. No. 6,166,302, and U.S. Pat. No. 5,380,831, herein incorporated by reference.

### Molecular Confirmation and Detection

Methods of confirming the presence of a polynucleotide insertion within the genome of a plant are known in the art. For example the detection of the polynucleotide insertion within the genome of a plant can be achieved, for example, by the polymerase chain reaction (PCR). The PCR detection is done by the use of two oligonucleotide primers flanking the polymorphic segment of the polymorphism followed by DNA amplification. This step involves repeated cycles of heat denaturation of the DNA followed by annealing of the primers to their complementary sequences at low temperatures, and extension of the annealed primers with DNA polymerase. Size separation of DNA fragments on agarose or polyacrylamide gels following amplification, comprises the major part of the methodology. Such selection and screening methodologies are well known to those skilled in the art. Molecular confirmation methods that can be used to identify transgenic plants are known to those with skill in the art. Several exemplary methods are further described below.

Molecular Beacons have been described for use in sequence detection. Briefly, a FRET oligonucleotide probe is designed that overlaps the flanking genomic and insert DNA junction. The unique structure of the FRET probe results in it containing a secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe(s) to the target sequence results in the removal of the probe secondary structure and spatial separation of the fluorescent and quenching moieties. A fluorescent signal indicates the presence of the flanking genomic/transgene insert sequence due to successful amplification and hybridization. Such a molecular beacon assay for detection of as an amplification reaction is an embodiment of the subject disclosure.

Hydrolysis probe assay, otherwise known as TAQMAN® (Life Technologies, Foster City, Calif.), is a method of detecting and quantifying the presence of a DNA sequence. Briefly, a FRET oligonucleotide probe is designed with one oligo within the transgene and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization. Such a hydrolysis probe assay for detection of as an amplification reaction is an embodiment of the subject disclosure.

KASPar® assays are a method of detecting and quantifying the presence of a DNA sequence. Briefly, the genomic DNA sample comprising the integrated gene expression cassette polynucleotide is screened using a polymerase chain reaction (PCR) based assay known as a KASPar® assay system. The KASPar® assay used in the practice of the subject disclosure can utilize a KASPar® PCR assay mixture which contains multiple primers. The primers used in the PCR assay mixture can comprise at least one forward primers and at least one reverse primer. The forward primer contains a sequence corresponding to a specific region of the DNA polynucleotide, and the reverse primer contains a sequence corresponding to a specific region of the genomic sequence. In addition, the primers used in the PCR assay mixture can comprise at least one forward primers and at least one reverse primer. For example, the KASPar® PCR assay mixture can use two forward primers corresponding to two different alleles and one reverse primer. One of the forward primers contains a sequence corresponding to specific region of the endogenous genomic sequence. The second forward primer contains a sequence corresponding to a specific region of the DNA polynucleotide. The reverse primer contains a sequence corresponding to a specific region of the genomic sequence. Such a KASPar® assay for detection of an amplification reaction is an embodiment of the subject disclosure.

In some embodiments the fluorescent signal or fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye.

In other embodiments the amplification reaction is run using suitable second fluorescent DNA dyes that are capable of staining cellular DNA at a concentration range detectable by flow cytometry, and have a fluorescent emission spectrum which is detectable by a real time thermocycler. It should be appreciated by those of ordinary skill in the art that other nucleic acid dyes are known and are continually being identified. Any suitable nucleic acid dye with appropriate excitation and emission spectra can be employed, such as YO-PRO-1®, SYTOX Green®, SYBR Green I®, SYTO11®, SYTO12®, SYTO13®, BOBO®, YOYO®, and TOTO®.

In further embodiments, Next Generation Sequencing (NGS) can be used for detection. As described by Brautigma et al., 2010, DNA sequence analysis can be used to determine the nucleotide sequence of the isolated and amplified fragment. The amplified fragments can be isolated and sub-cloned into a vector and sequenced using chain-terminator method (also referred to as Sanger sequencing) or Dye-terminator sequencing. In addition, the amplicon can be sequenced with Next Generation Sequencing. NGS technologies do not require the sub-cloning step, and multiple sequencing reads can be completed in a single reaction. Three NGS platforms are commercially available, the Genome Sequencer FLX™ from 454 Life Sciences/Roche, the Illumina Genome Analyser™ from Solexa and Applied Biosystems' SOLID™ (acronym for: ‘Sequencing by Oligo Ligation and Detection’). In addition, there are two single molecule sequencing methods that are currently being developed. These include the true Single Molecule Sequencing (tSMS) from Helicos Bioscience™ and the Single Molecule Real Time™ sequencing (SMRT) from Pacific Biosciences.

The Genome Sequencher FLX™ which is marketed by 454 Life Sciences/Roche is a long read NGS, which uses emulsion PCR and pyrosequencing to generate sequencing reads. DNA fragments of 300-800 bp or libraries containing fragments of 3-20 kb can be used. The reactions can produce over a million reads of about 250 to 400 bases per run for a total yield of 250 to 400 megabases. This technology produces the longest reads but the total sequence output per run is low compared to other NGS technologies.

The Illumina Genome Analyser™ which is marketed by Solexa™ is a short read NGS which uses sequencing by synthesis approach with fluorescent dye-labeled reversible terminator nucleotides and is based on solid-phase bridge PCR. Construction of paired end sequencing libraries containing DNA fragments of up to 10 kb can be used. The reactions produce over 100 million short reads that are 35-76 bases in length. This data can produce from 3-6 gigabases per run.

The Sequencing by Oligo Ligation and Detection (SOLID) system marketed by Applied Biosystems™ is a short read technology. This NGS technology uses fragmented double stranded DNA that are up to 10 kb in length. The system uses sequencing by ligation of dye-labelled oligonucleotide primers and emulsion PCR to generate one billion short reads that result in a total sequence output of up to 30 gigabases per run.

The tSMS of Helicos Bioscience™ and SMRT of Pacific Biosciences™M apply a different approach which uses single DNA molecules for the sequence reactions. The tSMS Helicos™ system produces up to 800 million short reads that result in 21 gigabases per run. These reactions are completed using fluorescent dye-labelled virtual terminator nucleotides that is described as a ‘sequencing by synthesis’ approach.

The SMRT Next Generation Sequencing system marketed by Pacific Biosciences™ uses a real time sequencing by synthesis. This technology can produce reads of up to 1,000 bp in length as a result of not being limited by reversible terminators. Raw read throughput that is equivalent to one-fold coverage of a diploid human genome can be produced per day using this technology.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments of the inventions recited herein and in the specification rather than the specific examples that are exemplified below. All cited patents and publications referred to in this application are herein incorporated by reference in their entirety, for all purposes, to the same extent as if each were individually and specifically incorporated by reference.

### EXAMPLES

The following are examples of specific embodiments of some aspects of the invention. The examples are offered for illustrative purposes only, and are not intended to limit the scope of the invention in any way. Efforts have been made to ensure accuracy with respect to numbers used (e.g., amounts, temperatures, etc.), but some experimental error and deviation should, of course, be allowed for.

### Example 1: Genome Editing Tools for DNA Target Site Cleavage and Promoter Activation

In this example, methods for producing genome editing tools capable of cleaving a genomic DNA target site and activating a promoter inserted at the cleavage site in a cell, particularly a plant cell, are described.

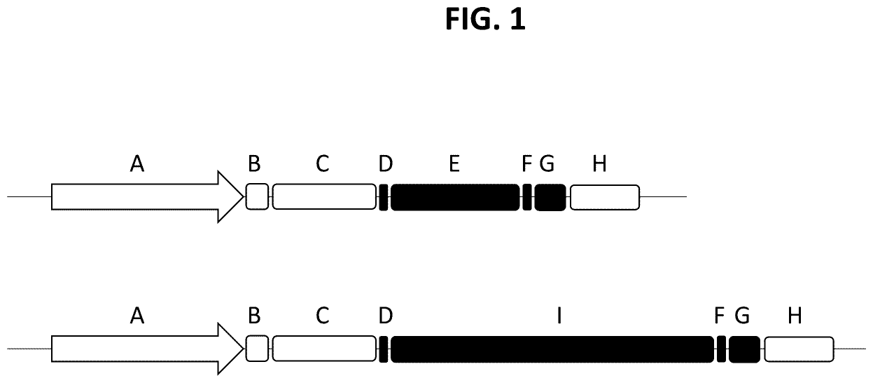

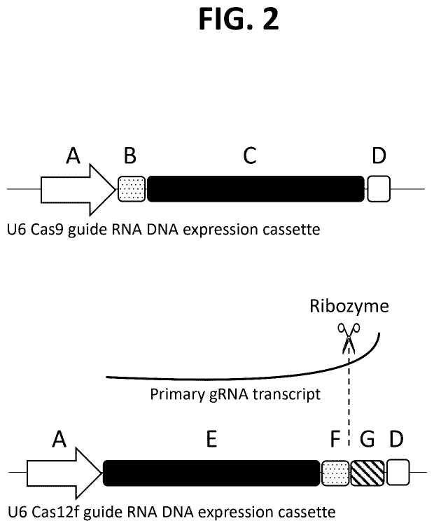

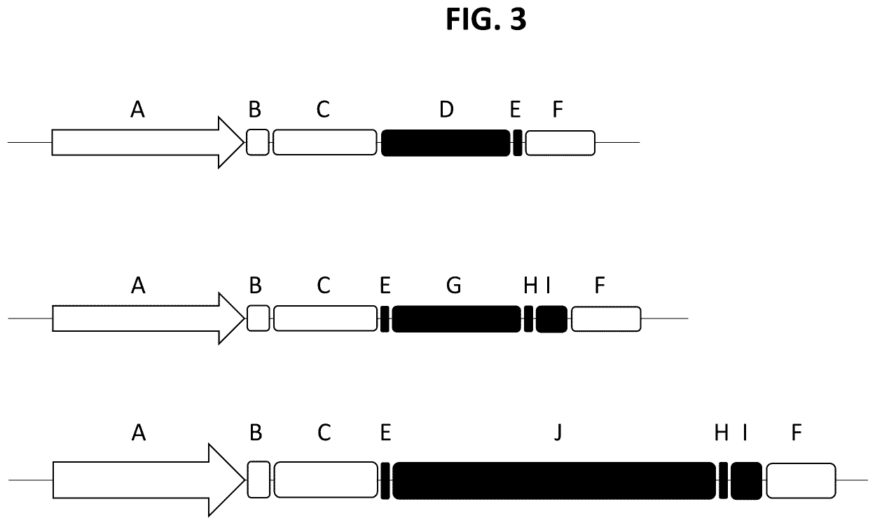

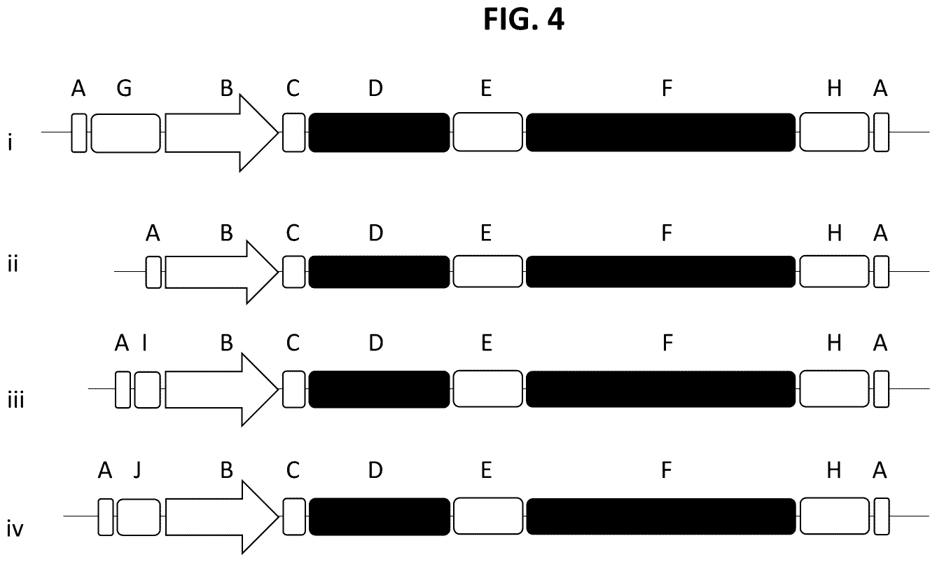

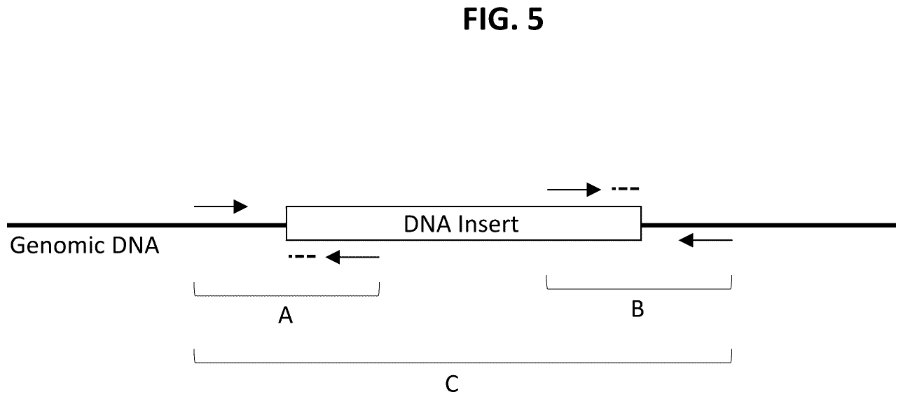

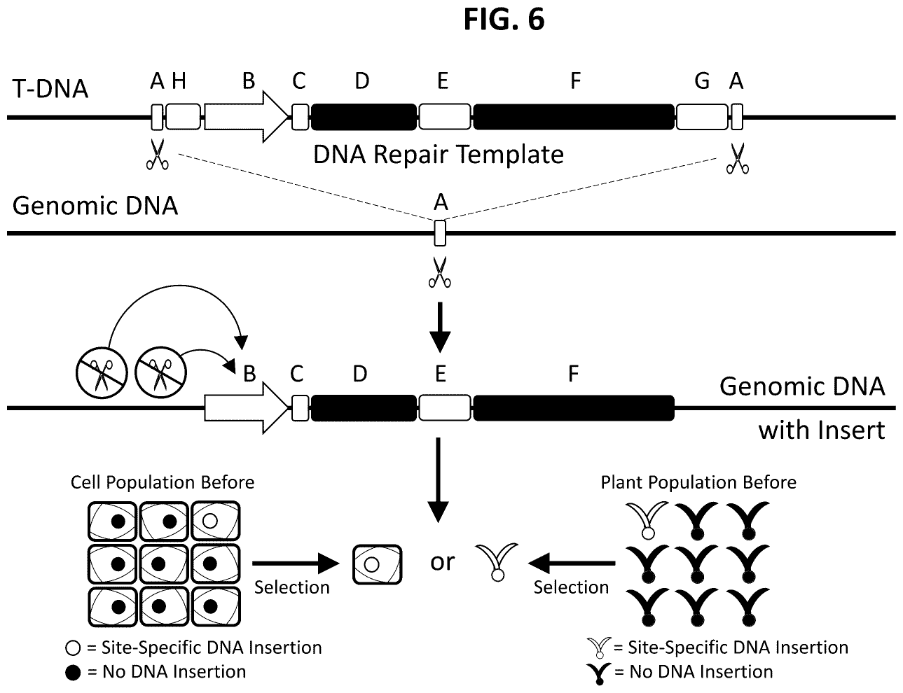

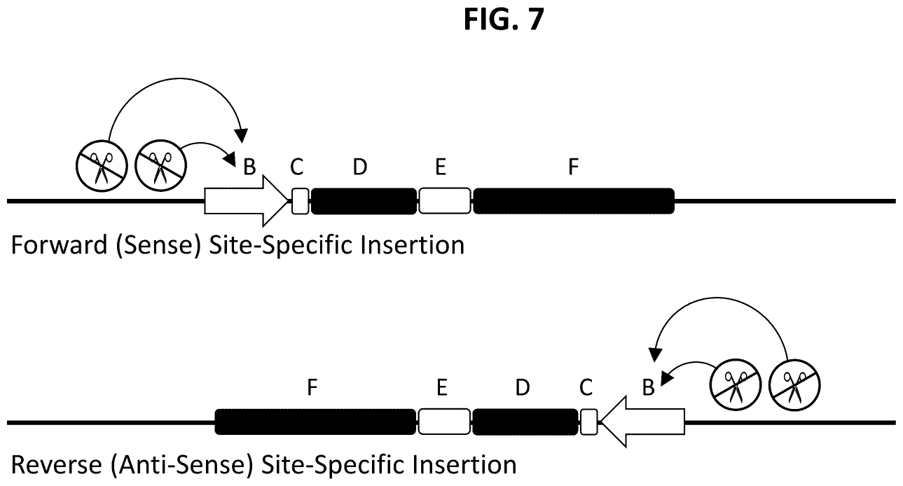

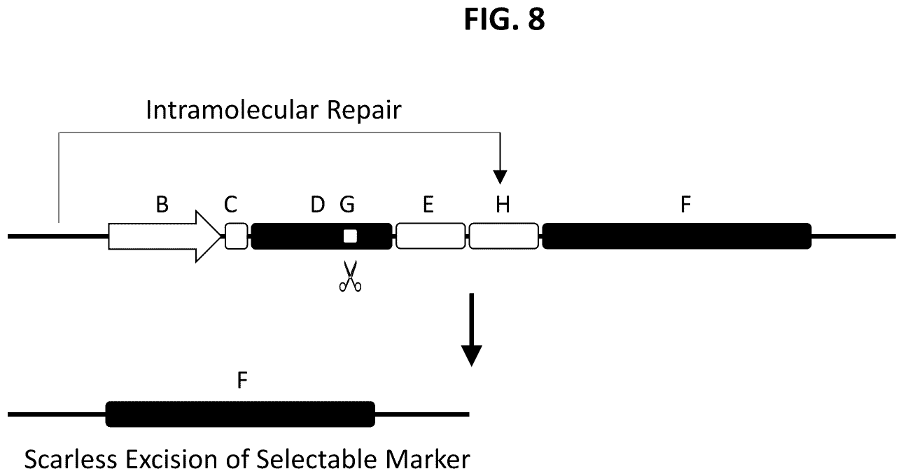

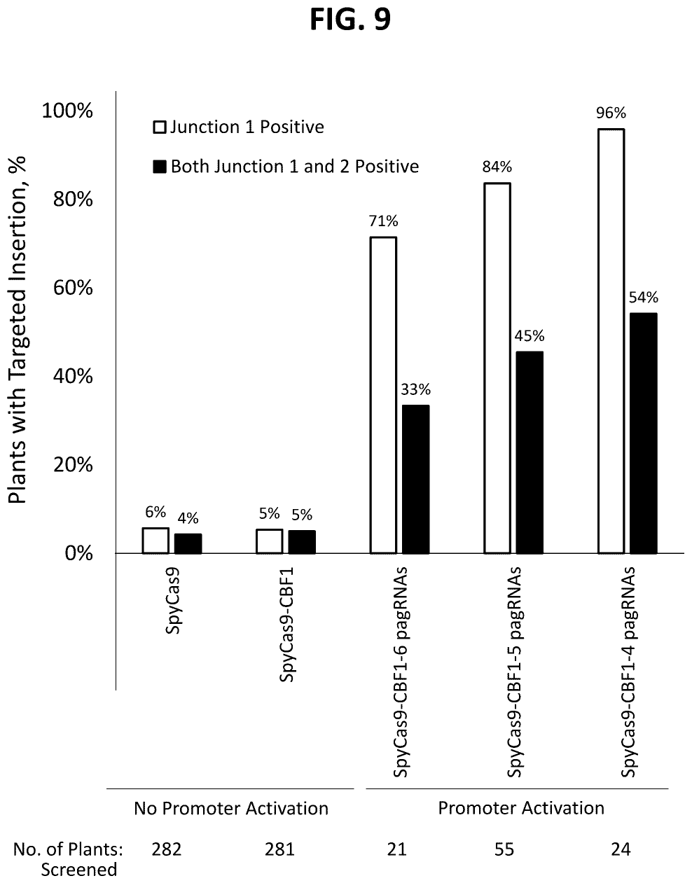

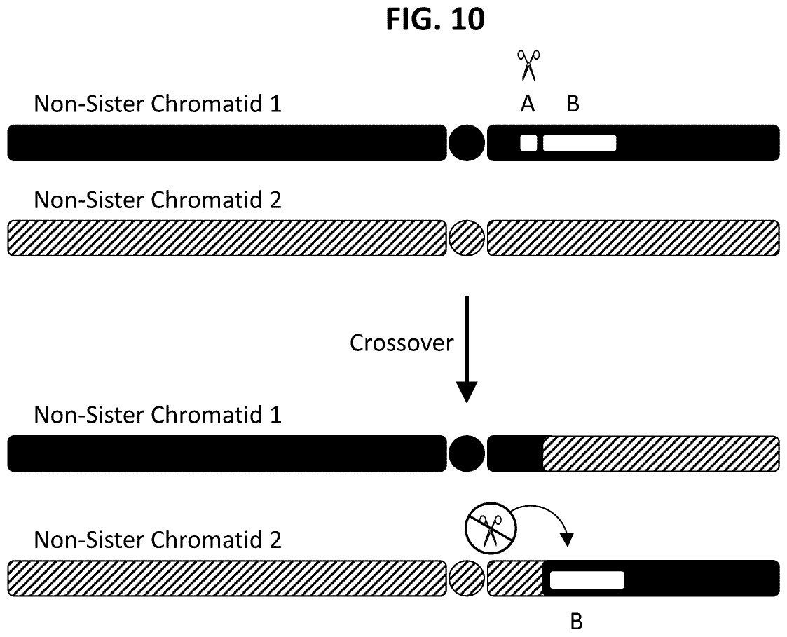

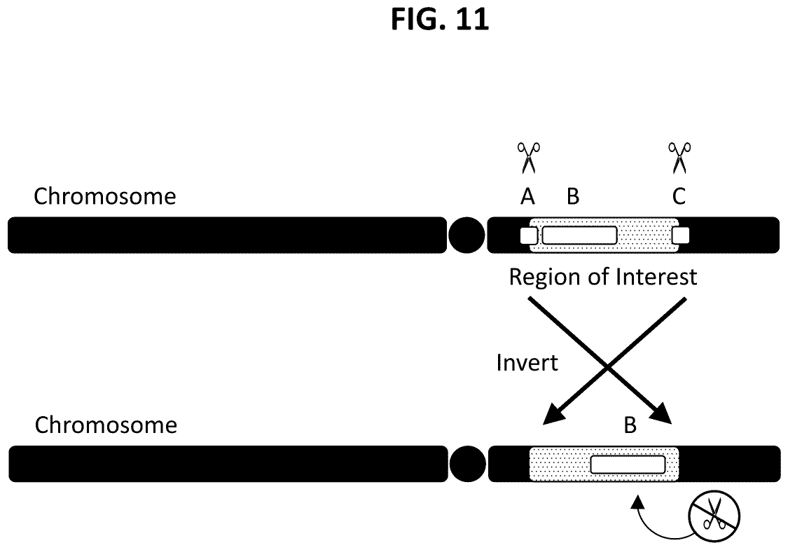

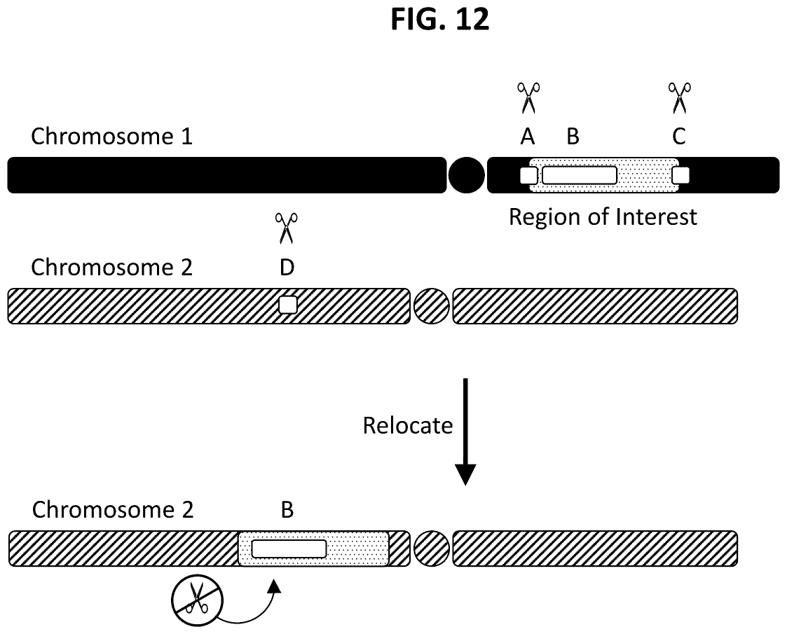

In one method, a Clustered Regularly Interspaced Short Pallindromic Repeat (CRISPR)-associated (Cas) endonuclease (although other RNA guided nucleases could be used, for example but not limited to transposon-associated protein B (TnpB), a Fanzor, or a HERMES (Karvelis et al. (2021) Nature. 599:692-696, Saito et al. (2023) Nature. available at doi.org/10.1038/s41586-023-06356-2, or Jiang et al. (2023) bioRxiv. doi: https://doi.org/10.1101/2023.06.13.544871)) was engineered with dual functionality capable of either DNA target cleavage or DNA target transcriptional activation. First, sequences encoding a Cas endonuclease (for example but not limited to the Cas9 from Streptococcus pyogenes (Spy) (SEQ ID NO:1), Cas12f from Syntrophomonas palmitica (Spa) or engineered variants thereof (SEQ ID NO:2; SEQ ID NO:3; SEQ ID NO:4), or Cas12f from Acidibacillus sulfuroxidans (Asu) or engineered variants thereof (SEQ ID NO:5 and SEQ ID NO:6)), transcriptional activation domain (TAD) (for example but not limited to the C-terminal acidic plant transcriptional activation domain from the Arabidopsis cold binding factor 1 (CFB1) protein (SEQ ID NO:7), and a nuclear localization sequence (NLS) (for example but not limited to the monopartite Simian virus 40 nuclear localization signal (mNLS) (PKKKRKV) or a bi-partite NLS from the VirD2 protein from Agrobacterium tumefaciens (bNLS) (SEQ ID NO:8)) were codon optimized using Zea mays codon tables and GC content adjusted using standard techniques known in the art and repetitive sequences and gene destabilizing features such as miniature inverted-repeat transposable elements (MITEs) removed. Next, optimized sequences encoding the TAD and NLS were appended in frame to the ends of the optimized sequence encoding the Cas endonuclease in a mNLS-Cas-bNLS-CBF1 configuration although other configurations can be utilized. The resulting gene was then synthesized (GenScript, USA) and cloned by restriction enzyme digestion and ligation into a Gateway compatible plasmid DNA containing a polymerase II promoter and terminator. For expression in maize, this includes a Zea mays Ubiquitin (UBI) promoter and terminator. To further enhance expression, a 5′ untranslated region (UTR) (for example but not limited to the maize UBI 5′ UTR) and additional introns (for example the UBI Zea mays intron 1 and potato ST-LSI intron 2) were used although other introns would work. Examples of a maize cell optimized Cas DNA expression construct are illustrated in FIG. 1.

The small RNAs that guide the mNLS-Cas-bNLS-CBFI chimeric protein described herein to a DNA target (referred to herein as guide RNAs (gRNAs)) were next engineered to specify either target cleavage or promoter activation. For this, the length of the variable targeting (VT) domain of the gRNA that serves to direct mNLS-Cas-bNLS-CBF1 to its DNA target by base pairing with one strand of the DNA target site adjacent to a suitable protospacer or target adjacent motif (PAM or TAM) (5′-TTC-3′ for SpaCas12f1, 5′-TTN-3′ for AsuCas12f1, and 5′-NGG-3′ for SpyCas9) was modulated. For DNA target cleavage, a VT length of 17-20 nts was utilized while for DNA target binding and promoter activation a VT length of 10-15 nts was used similar to that described earlier (Dahlman et al. (2015) Nat. Biotechnol. 33: 1159-1161). The 5′ and 3′ ends of sequences encoding the gRNAs were next operably linked to a U6 polymerase III promoter and terminator (TTTTTTTT). The resulting U6 promoter-gRNA-U6 terminator expression cassette was then synthesized (GenScript, USA) and cloned into a Gateway compatible plasmid DNA by restriction enzyme digestion and ligation. To promote optimal transcription of the gRNA from the U6 promoter, a G nucleotide was added to the 5′ end of the sequence to be transcribed. A ribozyme motif (Gao et al. (2014) J. Integr. Plant Biol. 56:343-349)), RNase P and Z cleavage sites (Xie et al. (2015) Proc. Natl. Acad. Sci. USA. 112:3570-3575), and/or Csy4 (Cas6 or CasE) ribonuclease recognition site (Tsai et al. (2014) Nat. Biotechnol. 32:569-576) can also be used to aid in the maturation of the gRNA transcript following transcription. Moreover, the RNA processing provided by these strategies can also be used to express multiple gRNAs from either a single polymerase II or III promoter (Gao et al. (2014), Xie et al. (2015), and Tsai et al. (2014)). Examples of the maize optimized Cas gRNA expression constructs are illustrated in FIG. 2.

In another method, two orthogonal genome editing tools are used, one to cut a DNA target site and the other to activate a promoter. This may encompass an RNA guided editing tool, those that don't require a gRNA (for example but not limited to a Zinc Finger nuclease (Urnov et al. (2010) Nature Reviews Genetics. 11: 636-636), TALE nuclease (Becker et al. (2021) Gene and Genome Editing. 2: 100007), or Meganuclease (Zekonyte et al. (2021) Nature Communications. 12: 3210)), or combinations of different tools. For this, sequences encoding the genome editing enzyme are conditioned for expression as described above. Next, for the genome editing protein destined to activate a promoter, an alanine or other suitable amino acid is substituted into one or more key residues required for DNA target cleavage or in the case of a Zinc finger or TALE nuclease, the FokI nuclease domain is removed. This enables the genome editing protein to bind but not cleave a DNA site. As described above, sequences encoding a TAD and NLS are then linked in-frame with the sequence encoding the nuclease dead (d) protein. The sequence encoding the genome editing nuclease is fused in-frame with only the sequence encoding the NLS. Both chimeric genes are then synthesized (GenScript, USA) and cloned using restriction enzyme digestion and ligation into a Gateway compatible plasmid DNA containing a promoter, 5′ UTR, introns, and terminator as described above. gRNAs are optimized for expression as described above except gRNAs with only a 17-20 nt length VT are used. Example expression cassettes are shown in FIG. 3.

### Example 2: DNA Repair Template for Site-Specific Insertion

In this example, methods for producing DNA repair templates for site-specific insertion into a genomic DNA target of a cell, particularly a plant cell, are described.

In one method, the 5′ and 3′ ends of the DNA sequence to be inserted (for example but not limited to a gene conferring a desirable trait) were appended with additional DNA sequences. This included a minimal, sub-optimal, or non-functional promoter (for example but not limited to a minimal 35S cauliflower mosaic virus promoter), a 5′ UTR (for example but not limited to the 5′ UTR from the tobacco mosaic virus (TMV)), a selectable marker (for example but not limited to sequences encoding neomycin phosphotransferase II (NPTII) (SEQ ID NO: 60), plant phosphomannose isomerase (PMI) (SEQ ID NO:61), Wuschel2 (Wus2) (SEQ ID NO: 62), Baby Boom (Bbm) (SEQ ID NO:63), the reef coral red fluorescent protein from Discosoma sp. (DsRed) (SEQ ID NO:64), or a fusion between a maize-optimized phosphinothricin acetyl transferase (moPAT) and DsRed (moPAT-DsRed) protein (SEQ ID NO: 65)), and a terminator (for example but not limited to the T28 terminator from Oryza sativa), optionally a DNA sequence homologous to that flanking either one or both sides of the genomic DNA target cleavage site, and optionally one or more DNA cleavage sites. The resulting DNA repair template was then synthesized (GenScript, USA) and restriction enzyme ligated into a Gateway compatible plasmid DNA using methods known in the art. Schematics of DNA repair templates for site specific insertion into a genomic DNA target of a cell are shown in FIG. 4.

### Example 3: Transformation of Components for Site-Specific DNA Insertion

In this example, methods for introducing genome editing tool expression cassette(s) and DNA repair template for site-specific insertion into a genomic DNA target in a cell, particularly a plant cell, are described.

Although other transformation methods for example but not limited to Biolistic, Ensifer-based, nanoparticle-mediated or approaches utilizing protoplasts may be used (Rathore et al. (2019) Transgenic Plants: Methods and Protocols. New York, NY: Springer New York, 37-48, Wang et al. (2019) Molecular Plant. 12, 1037-1040, Rhodes et al. (1988) Science. 240, 204-207 and Golovkin et al. (1993) Plant Science. 90, 41-52), the genome editing tool expression cassette(s) and DNA repair template were co-delivered on a single transfer DNA (T-DNA) into maize immature embryos using Agrobacterium as described earlier (Lowe et al. (2018) In vitro cellular & developmental biology—Plant. 54, 240-252). Briefly, the optimized genome modifying tool expression cassette(s) and DNA repair template were Gateway cloned into a T-DNA optionally already containing genes encoding the morphogenic transcription factors Bbm and Wus2 operably linked to Zm-PLTP and Axig1 promoters, respectively, if not utilized in the repair template. The resulting T-DNA was then placed into LBA4404, a thymine auxotrophic strain of Agrobacterium tumefaciens, containing Vir9, a separate plasmid encoding the Bo542 virulence genes (US20170121722A1 and WO 2017/078836). The resulting Agrobacterium strain was then used to transform nine-to-ten-day old immature maize embryos (approximately 2 mm in size) by submersion in 700A liquid media containing the strain at an optical density of 0.7 at 550 nm for 5 min. and then removed from the media and placed on solid co-cultivation medium overnight at 21° C. in the dark. Following T-DNA delivery, embryos were transferred to resting media, 13266R, and grown in the dark at 28° C. for 5-7 days. Next, coleoptiles were removed, and embryos transferred to selection media for 11-16 days and kept in the dark at 28° C. They were then moved to maturation media and incubated for 14-25 days in the dark at 28° C. and then subject to light for 2-5 days. Next, they were moved to rooting media and incubated at 26-28° C. under light for 14-28 days refreshing media as needed. To foster conditions optimal for genome editing tool activity, transformed tissue was incubated at 28° C., standard for tissue culture, or at a range of temperatures lower or higher than 28° C.

### Example 4: Detection of Site-Specific DNA Insertion in a Cell

In this example, methods for detecting a site-specific DNA insertion in a cell, particularly a plant cell, are described.

In one method, PCR was used to detect a site-specific DNA insertion in a plant cell as described previously (Peterson et al. (2021) Plant Biotechnol. J. 19: 2000-2010). Briefly, primers were designed and synthesized (Integrated DNA Technologies, USA) to amplify each junction created by the site-specific insertion of the DNA fragment (FIG. 5). Next, T0 plants were sampled (V2 or V3 leaf punches) and DNA extracted with a Synergy 2.0 Plant DNA Extraction Kit (Ops Diagnostics, USA). Quantitative PCR (qPCR) was next utilized to screen for T0 plants containing a putative insertion. In this case, a TaqMan probe was also designed, synthesized (Integrated DNA Technologies, USA), and used along with the aforementioned primers (FIG. 5). Plants with a qPCR positive signal relative to the negative control (untransformed wildtype genomic DNA) were next subject to additional analysis. This included PCR amplification and fragment size analysis using agarose gel electrophoresis of each genomic and insert junction. T0 plants demonstrating a correct size amplification product at both junctions can also be subject to long-PCR. For this, forward and reverse primers outside of the DNA insert in the flanking genomic regions are used to amplify across the entire DNA insert and the presence of a PCR product of the expected size used to confirm the presence of an intact site-specific DNA insertion (FIG. 5).

### Example 5: Selecting for Site-Specific DNA Insertion

In this example, methods for selecting for DNA inserted at or near a genome editing tool DNA target cleavage site by activating a promoter are described.

In one method, the fraction of cells or plants that contain a site-specific DNA insertion were enriched for by activating a promoter operably linked to a selectable marker (FIG. 6). For this, a genome modifying tool was used to cleave a DNA target site in the presence of a DNA repair template containing a desirable trait gene(s) and a minimal, sub-optimal, or non-functional promoter driving the expression of a selectable marker (FIG. 6). Then, a genome modifying tool capable of binding but not cutting one or more DNA target sites was directed to a genomic region flanking the cleaved DNA target such that it activates expression of the selectable marker only in the cells or plants containing the site-specific DNA insertion (FIG. 6). Next, a selective agent (chemical or visual) was applied to enrich for cells or plants that contain the site-specific DNA insertion. Using this approach, the directionality of the inserted DNA may also be controlled by the positioning of the promoter activation target sites (FIG. 7). If positioned 5′ of the cleavage site, DNA insertions will occur in a forward or sense direction and, if positioned 3′ of the cleavage site, they will insert in a reverse or anti-sense orientation (FIG. 7). Different selectable markers can also be used concurrently to enable multiplexed DNA insertion. If desired, the selectable marker can be excised following selection using an inducible site-specific recombinase (Cre) as described earlier for the removal of morphogenic genes (Wang et al. (2020) Frontiers in Plant Science. 11: 1298). Alternatively, a sequence homologous to the genomic DNA flanking the cleavage site can be placed between the selectable marker and desirable trait gene (FIG. 8). And then, by targeting the intervening sequence for cleavage, intramolecular repair between the directly repeated homologous sequences as described earlier when reconstituting the β-glucuronidase (GUS) gene (Orel et al. (2003) Plant J. 35: 604-612) can be used to excise the selectable marker gene in a scarless fashion (FIG. 8).

### Example 6: Efficient Recovery of Site-Specific DNA Insertion in Plants

In this example, the methods described herein are utilized to improve the recovery of targeted DNA insertion in plants.

Transformation was carried-out as described above using Agrobacterium and T0 maize plantlets expressing DsRed identified using an Xite Fluorescence Flashlight System (NIGHTSEA, USA). After sampling from the red fluorescing plants, genomic DNA was extracted, and evaluated for the presence of site-specific gene insertion as described in Example 4. Controls utilized both mNLS-SpyCas9-bNLS and mNLS-SpyCas9-bNLS-CBF1 and included only one gRNA capable of cleaving the genomic target site and excising the repair template. Promoter activation experiments used mNLS-SpyCas9-bNLS-CBF1, one gRNA to cleave the genomic target site and excise the repair template, and either four, five, or six promoter activating gRNAs (pagRNAs). DNA repair templates for the controls are depicted in FIG. 4i. For the activation experiments, four, five, and six pagRNAs were used in combination with the DNA repair templates shown in FIGS. 4iv, 4iii, and 4ii, respectively.

As shown in FIG. 9, 71%, 84%, and 96% of the T0 plantlets when using six, five, and four pagRNAs, respectively, were observed to be PCR positive for Junction 1, the unique junction generated by the insertion of the minimal promoter and selectable marker gene into the SpyCas9 genomic cleavage site. Without activation, the frequency of plants positive for junction 1 was between 5 and 6% (FIG. 9). Next, to assess the integrity of the targeted insertion, the frequency of plants containing both Junction 1 and 2 were determined. As shown in FIG. 9, roughly half of the Junction 1 positive plants from the activation experiments were also positive for the insertion at Junction 2 producing 33%, 45%, and 54% of plants with a targeted gene insertion predicted to be intact. Altogether, a 7 to 14-fold enhancement in the recovery of targeted gene insertion was observed relative to experiments that did not utilize promoter activation.

### Example 7: Use of Site-Specific DNA Insertion to Select for Chromosomal DNA Crossover, Inversion, or Relocation

In this example, methods for selecting for chromosomal DNA crossover, inversion, or relocation at or near a genome modifying tool DNA target cleavage site are described.

In one method, a minimal, suboptimal, or non-functional promoter operably linked to a selectable marker (SM) is first inserted at the boundary of a chromosomal region destined for crossover, inversion, or relocation using the methods described herein. For a crossover, a region 5′ of the minimal, suboptimal, or non-functional promoter (MSNP) is next targeted for genome modifying tool cleavage (FIG. 10). In the case of an inversion, the region 5′ of the MSNP as well as the distal boundary (DB) of the chromosomal region of interest are targeted for cleavage (FIG. 11). For relocation, the region 5′ of the MSNP, the DB, and the new chromosomal location are targeted for cleavage (FIG. 12). Next, genome modification tools designed to activate selectable marker transcription are utilized to select for the desired DNA repair outcome (FIGS. 10-12). As described in Example 5, the selectable expression cassette can be excised if desired.

1. A method of editing a genome of a target cell to comprise a donor polynucleotide, the method comprising:
(a) Inserting a donor polynucleotide within a genomic target site, wherein the donor polynucleotide comprises a gene expression cassette comprising a non-functional promoter operably linked to a coding sequence such that the coding sequence is either not expressed or expressed at a low level;
(b) Binding a site specific-promoter activation complex to a genomic region upstream of the inserted donor polynucleotide, wherein the site specific-promoter activation complex functions to drive the non-functional promoter to express a protein from the coding sequence;
(c) Expressing the coding sequence at a higher level;
(d) Selecting for the target cell that expresses the coding sequence; and
(e) Obtaining the target cell that expresses the coding sequence.

2. The method of claim 1, wherein the coding sequence comprises an agronomic trait, wherein the agronomic trait is selected from the group consisting of an insecticidal resistance trait, herbicide tolerance trait, nitrogen use efficiency trait, water use efficiency trait, nutritional quality trait, DNA binding trait, small RNA trait, selectable marker trait, or any combination thereof.

3. The method of claim 1, wherein the coding sequence comprises a selectable marker.

4. The method of claim 4, wherein the selectable marker is selected from the group of nptII, pat, bar, dsm-2, ahas, gox, gat, gus, a fluorescent protein, or any combination thereof.

5. The method of claim 1, wherein the coding sequence confers resistance to a herbicide or an antibiotic.

6. The method of claim 1, wherein the non-functional promoter comprises a minimal promoter.

7. The method of claim 6, where in the minimal promoter comprises a TATA box, a CAAT box, a transcription start site, an RNA polymerase binding site, or any combination thereof.

8. The method of claim 1, wherein the non-functional promoter drives low levels of the coding sequence.

9. The method of claim 1, wherein the site specific-promoter activation complex comprises a site-specific binding protein operably linked to at least one activation domain.

10. The method of claim 9, wherein the site-specific binding protein is a CRIPSR protein, zinc finer protein, or a TALEN protein.

11. The method of claim 10, wherein the CRISPR protein is mutagenized to inactivate nuclease activity.

12. The method of claim 9, wherein the activation domain is RTA, TFIIA, TFIIB, TFIID, TFIIE, TFIIF, TFIIH, VP64, VP16, VP160, GAL4, EDLL, ERF2, CBF1, ORCA2, DREB1A, LEAFY, or any combination thereof.

13. The method of claim 1, wherein the site specific-promoter activation complex is bound within proximity to the non-functional promoter.

14. The method of claim 1, wherein the site specific-promoter activation complex is bound within 1-10,000 bp of the non-functional promoter.

15. The method of claim 1, wherein the coding sequence is expressed at a higher level that is 1%-100% higher than the initial low level expression of the coding sequence.

16. The method of claim 1 wherein the site specific-promoter activation complex drives expression of the coding sequence by at least 2-fold, at least 3-fold, at least 4-fold, at least 5-fold, at least 10-fold, at least 15-fold, at least 20-fold, at least 25-fold, or at least 50-fold higher than a coding sequence that is driven by only the non-functional promoter.

17. The method of claim 1, wherein multiple copies of the site-specific promoter activation complex are bound to the genomic region upstream of the inserted donor polynucleotide such that the site-specific promoter activation complex functions to drive the non-functional promoter.

18. The method of claim 1, wherein the cell is a plant cell.

19. The method of claim 18, wherein the plant cell is a monocotyledonous plant cell or a dicotyledonous plant cell.

20.-23. (canceled)
